(19) United States
(12) Patent Application Publication
(10) Pub. No.: US 2024/0206797 A1
(43) Pub. Date: Jun. 27, 2024

(54) TISSUE TREATMENT SYSTEM

(71) Applicant: Acutus Medical, Inc., Carlsbad, CA (US)

(72) Inventors: Derrick Chou, San Diego, CA (US); Timothy Corvi, San Diego, CA (US); Alex Asconeguy, Eagle, ID (US); Wilson William Good, Carlsbad, CA (US); R. Maxwell Flaherty, Topsfield, MA (US); J. Christopher Flaherty, Nottingham, NH (US); Joshua Krachman, Carlsbad, CA (US); Steven Yon, San Diego, CA (US); Jaume Coll-Font, Boston, MA (US)

(21) Appl. No.: 18/291,340

(22) PCT Filed: Jul. 27, 2022
(86) PCT No.: PCT/US2022/038464

§ 371 Date: Jan. 23, 2024

Related U.S. Application Data
(60) Provisional application No. 63/203,606, filed on Jul. 27, 2021.
(60) Provisional application No. 63/335,939, filed on Apr. 28, 2022.

(51) Int. Cl. A61B 5/349; A61B 5/339; A61B 5/367

(52) U.S. Cl. CPC A61B 5/349; A61B 5/339; A61B 5/367

(57) ABSTRACT

A cardiac information dynamic display system comprises one or more electrodes and a cardiac information console. The one or more electrodes record sets of electric potential data representing cardiac activity at a plurality of time intervals. The cardiac information console includes a signal processor and a user interface module. The signal processor calculates sets of cardiac activity data at the plurality of time intervals using the recorded sets of electric potential data. The cardiac activity data is associated with surface locations of one or more cardiac chambers. The user interface module displays information related to the cardiac activity data. The information is presented relative to a graphical representation of surfaces of the one or more cardiac chambers.

### RELATED APPLICATIONS

The present application claims priority to U.S. Provisional Patent Application Ser. No. 63/203,606, entitled “TISSUE TREATMENT SYSTEM”, filed Jul. 27, 2021, which is hereby incorporated by reference.

The present application claims priority to States Provisional Patent Application Ser. No. 63/335,939, entitled “TISSUE TREATMENT SYSTEM”, filed Apr. 28, 2022, which is hereby incorporated by reference.

The present application, while not claiming priority to, may be related to U.S. Provisional Application Ser. No. 63/226,040, entitled “Energy Delivery Systems With Lesion Index”, filed Jul. 27, 2021, which is hereby incorporated by reference.

The present application, while not claiming priority to, may be related to U.S. Provisional Application Ser. No. 63/260,234, entitled “Intravascular Atrial Fibrillation Treatment”, filed Aug. 13, 2021, which is hereby incorporated by reference.

The present application, while not claiming priority to, may be related to US national stage filing of Patent Cooperation Treaty Application No. PCT/US2022/016722, entitled “Energy Delivery Systems With Ablation Index”, filed Feb. 17, 2022, which claims priority to U.S. Provisional Application Ser. No. 63/150,555, entitled “Energy Delivery Systems With Ablation Index”, filed Feb. 17, 2021, each of which is hereby incorporated by reference.

The present application, while not claiming priority to, may be related to U.S. application Ser. No. 16/335,893, entitled “Ablation System with Force Control”, filed Mar. 22, 2019, which is a 35 USC 371 national stage filing of Patent Cooperation Treaty Application No. PCT/US2017/056064, entitled “Ablation System with Force Control”, filed Oct. 11, 2017, which claims priority to U.S. Provisional Application Ser. No. 62/406,748, entitled “Ablation System with Force Control”, filed Oct. 11, 2016, and U.S. Provisional Application Ser. No. 62/504,139, entitled “Ablation System with Force Control”, filed May 10, 2017, each of which is hereby incorporated by reference.

The present application, while not claiming priority to, may be related to U.S. application Ser. No. 16/097,955, entitled “Cardiac Information Dynamic Display System and Method”, filed Oct. 31, 2018, which is a 35 USC 371 national stage filing of Patent Cooperation Treaty Application No. PCT/US2017/030915, entitled “Cardiac Information Dynamic Display System and Method”, filed May 3, 2017, which claims priority to U.S. Provisional Application Ser. No. 62/331,351, entitled “Cardiac Information Dynamic Display System and Method”, filed May 3, 2016, each of which is hereby incorporated by reference.

The present application, while not claiming priority to, may be related to U.S. patent application Ser. No. 16/861,814, entitled “Catheter System and Methods of Medical Uses of Same, including Diagnostic and Treatment Uses for the Heart”, filed Apr. 29, 2020, which is a continuation of U.S. Pat. No. 10,667,753, entitled “Catheter System and Methods of Medical Uses of Same, Including Diagnostic and Treatment Uses for the Heart”, filed Jun. 19, 2018, which is a continuation of U.S. Pat. No. 10,004,459, entitled “Catheter System and Methods of Medical Uses of Same, Including Diagnostic and Treatment Uses for the Heart”, filed Feb. 20, 2015, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/242,810, entitled “Expandable Catheter Assembly with Flexible Printed Circuit Board (PCB) Electrical Pathways”, filed Jan. 8, 2019, which is a continuation of U.S. Pat. No. 10,201,311, entitled “Expandable Catheter Assembly with Flexible Printed Circuit Board (PCB) Electrical Pathways”, filed Jul. 23, 2015, which is a 35 USC 371 national stage filing of Patent Cooperation Treaty Application No. PCT/US2014/015261, entitled “Expandable Catheter Assembly with Flexible Printed Circuit Board (PCB) Electrical Pathways”, filed Feb. 7, 2014, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, each of which is hereby incorporated by reference.

The present application, while not claiming priority to, may be related to U.S. patent application Ser. No. 16/533,028, entitled “Method and Device for Determining and Presenting Surface Charge and Dipole Densities on Cardiac Walls”, filed Aug. 6, 2019, which is a continuation of U.S. Pat. No. 10,413,206, entitled “Method and Device for Determining and Presenting Surface Charge and Dipole Densities on Cardiac Walls”, filed Jun. 21, 2018, which is a continuation of U.S. Pat. No. 10,376,171, entitled “Method and Device for Determining and Presenting Surface Charge and Dipole Densities on Cardiac Walls”, filed Feb. 17, 2017, which is a continuation of U.S. Pat. No. 9,610,024, entitled “Method and Device for Determining and Presenting Surface Charge and Dipole Densities on Cardiac Walls”, filed Sep. 25, 2015, which is a continuation of U.S. Pat. No. 9,167,982, entitled “Method and Device for Determining and Presenting Surface Charge and Dipole Densities on Cardiac Walls”, filed Nov. 19, 2014, which is a continuation of U.S. Pat. No. 8,918,158, entitled “Method and Device for Determining and Presenting Surface Charge and Dipole Densities on Cardiac Walls”, filed Feb. 25, 2014, which is a continuation of U.S. Pat. No. 8,700,119, entitled “Method and Device for Determining and Presenting Surface Charge and Dipole Densities on Cardiac Walls”, filed Apr. 8, 2013, which is a continuation of U.S. Pat. No. 8,417,313, entitled “Method and Device for Determining and Presenting Surface Charge and Dipole Densities on Cardiac Walls”, filed Feb. 3, 2009, which is a 35 USC 371 national stage filing of PCT Application No. PCT/CH2007/000380, entitled “Method and Device for Determining and Presenting Surface Charge and Dipole Densities on Cardiac Walls”, filed Aug. 3, 2007, which claims priority to Swiss Patent Application No. 1251/06, filed Aug. 3, 2006, each of which is hereby incorporated by reference.

The present application, while not claiming priority to, may be related to U.S. Pat. No. 11,116,438, entitled “Device and Method for the Geometric Determination of Electrical Dipole Densities on the Cardiac Wall”, filed Sep. 12, 2019, which is a continuation of U.S. Pat. No. 10,463,267, entitled “Device and Method for the Geometric Determination of Electrical Dipole Densities on the Cardiac Wall”, filed Jan. 29, 2018, which is a continuation of U.S. Pat. No. 9,913,589, entitled “Device and Method for the Geometric Determination of Electrical Dipole Densities on the Cardiac Wall”, filed Oct. 25, 2016, which is a continuation of U.S. Pat. No. 9,504,395, entitled “Device and Method for the Geometric Determination of Electrical Dipole Densities on the Cardiac Wall”, filed Oct. 19, 2015, which is a continuation of U.S. Pat. No. 9,192,318, entitled “Device and Method for the Geometric Determination of Electrical Dipole Densities on the Cardiac Wall”, filed Jul. 19, 2013, which is a continuation of U.S. Pat. No. 8,512,255, entitled “Device and Method for the Geometric Determination of Electrical Dipole Densities on the Cardiac Wall”, filed Jul. 16, 2010, which is a 35 USC 371 national stage application of Patent Cooperation Treaty Application No. PCT/IB2009/000071, filed Jan. 16, 2009, entitled “A Device and Method for the Geometric Determination of Electrical Dipole Densities on the Cardiac Wall”, which claimed priority to Swiss Patent Application 00068/08 filed Jan. 17, 2008, each of which is hereby incorporated by reference.

The present application, while not claiming priority to, may be related to U.S. patent application Ser. No. 17/673,995, entitled “Device and Method for the Geometric Determination of Electrical Dipole Densities on the Cardiac Wall”, filed Feb. 17, 2022, which is a continuation of U.S. Pat. No. 11,278,209, entitled “Device and Method for the Geometric Determination of Electrical Dipole Densities on the Cardiac Wall”, filed Apr. 19, 2019, which is a continuation of U.S. Pat. No. 10,314,497, entitled “Device and Method for the Geometric Determination of Electrical Dipole Densities on the Cardiac Wall”, filed Mar. 20, 2018, which is a continuation of U.S. Pat. No. 9,968,268, entitled “Device and Method for the Geometric Determination of Electrical Dipole Densities on the Cardiac Wall”, filed Aug. 8, 2017, which is a continuation of U.S. Pat. No. 9,757,044, entitled “Device and Method for the Geometric Determination of Electrical Dipole Densities on the Cardiac Wall”, filed Sep. 6, 2013, which is a 35 USC 371 national stage filing of Patent Cooperation Treaty Application No. PCT/US2012/028593, entitled “Device and Method for the Geometric Determination of Electrical Dipole Densities on the Cardiac Wall”, filed Mar. 9, 2012, which claimed priority to U.S. Patent Provisional Application Ser. No. 61/451,357, filed Mar. 10, 2011, each of which is hereby incorporated by reference.

The present application, while not claiming priority to, may be related to US Design Patent No. 29/681,827, entitled “Set of Transducer-Electrode Pairs for a Catheter”, filed Feb. 28, 2019, which is a division of US Design Pat. No. D851,774, entitled “Set of Transducer-Electrode Pairs for a Catheter”, filed Feb. 6, 2017, which is a division of US Design Pat. No. D782,686, entitled “Transducer-Electrode Pair for a Catheter”, filed Dec. 2, 2013, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/111,538, entitled “Gas-Elimination Patient Access Device”, filed Aug. 24, 2018, which is a continuation of U.S. Pat. No. 10,071,227, entitled “Gas-Elimination Patient Access Device”, filed Jul. 14, 2016, which is a 35 USC 371 national stage filing of Patent Cooperation Treaty Application No. PCT/US2015/011312, entitled “Gas-Elimination Patient Access Device”, filed Jan. 14, 2015, which claims priority to U.S. Patent Provisional Application Ser. No. 61/928,704, entitled “Gas-Elimination Patient Access Device”, filed Jan. 17, 2014, which is hereby incorporated by reference.

The present application, while not claiming priority to, may be related to U.S. patent application Ser. No. 17/578,522, entitled “Cardiac Analysis User Interface System and Method”, filed Jan. 19, 2022, which is a continuation of U.S. Pat. No. 11,278,231, entitled “Cardiac Analysis User Interface System and Method”, filed Sep. 23, 2016, which is a 35 USC 371 national stage filing of Patent Cooperation Treaty Application No. PCT/US2015/022187, entitled “Cardiac Analysis User Interface System and Method”, filed Mar. 24, 2015, which claims priority to U.S. Patent Provisional Application Ser. No. 61/970,027, entitled “Cardiac Analysis User Interface System and Method”, filed Mar. 25, 2014, which is hereby incorporated by reference.

The present application, while not claiming priority to, may be related to U.S. patent application Ser. No. 17/063,901, entitled “Devices and Methods for Determination of Electrical Dipole Densities on a Cardiac Surface”, filed Oct. 6, 2020, which is a continuation of U.S. Pat. No. 10,828,011, entitled “Devices and Methods for Determination of Electrical Dipole Densities on a Cardiac Surface”, filed Mar. 2, 2016, which is a 35 USC 371 national stage filing of Patent Cooperation Treaty Application No. PCT/US2014/054942, entitled “Devices and Methods for Determination of Electrical Dipole Densities on a Cardiac Surface”, filed Sep. 10, 2014, which claims priority to U.S. Patent Provisional Application Ser. No. 61/877,617, entitled “Devices and Methods for Determination of Electrical Dipole Densities on a Cardiac Surface”, filed Sep. 13, 2013, which is hereby incorporated by reference.

The present application, while not claiming priority to, may be related to U.S. patent application Ser. No. 16/849,045, entitled “Localization System and Method Useful in the Acquisition and Analysis of Cardiac Information”, filed Apr. 15, 2020, which is a continuation of U.S. Pat. No. 10,653,318, entitled “Localization System and Method Useful in the Acquisition and Analysis of Cardiac Information”, filed Oct. 26, 2017, which is a 35 USC 371 national stage filing of Patent Cooperation Treaty Application No. PCT/US2016/032420, entitled “Localization System and Method Useful in the Acquisition and Analysis of Cardiac Information”, filed May 13, 2016, which claims priority to U.S. Patent Provisional Application Ser. No. 62/161,213, entitled “Localization System and Method Useful in the Acquisition and Analysis of Cardiac Information”, filed May 13, 2015, which is hereby incorporated by reference.

The present application, while not claiming priority to, may be related to U.S. patent application Ser. No. 15/569,231, entitled “Cardiac Virtualization Test Tank and Testing System and Method”, filed Oct. 25, 2017, which is a 35 USC 371 national stage filing of Patent Cooperation Treaty Application No. PCT/US2016/031823, filed May 11, 2016, which claims priority to U.S. Patent Provisional Application Ser. No. 62/160,501, entitled “Cardiac Virtualization Test Tank and Testing System and Method”, filed May 12, 2015, which is hereby incorporated by reference.

The present application, while not claiming priority to, may be related to U.S. patent application Ser. No. 17/735,285, entitled “Ultrasound Sequencing System and Method”, filed May 3, 2022, which is a continuation of to U.S. Patent Application Ser. No. 15/569,185, entitled “Ultrasound Sequencing System and Method”, filed Oct. 25, 2017, which is a 35 USC 371 national stage filing of Patent Cooperation Treaty Application No. PCT/US2016/032017, filed May 12, 2016, which claims priority to U.S. Patent Provisional Application Ser. No. 62/160,529, entitled “Ultrasound Sequencing System and Method”, filed May 12, 2015, which is hereby incorporated by reference.

The present application, while not claiming priority to, may be related to U.S. patent application Ser. No. 17/858,174, entitled “Cardiac Mapping System with Efficiency Algorithm”, filed Jul. 6, 2022, which is a Continuation application of U.S. patent application Ser. No. 16/097,959, entitled “Cardiac Mapping System with Efficiency Algorithm”, filed Oct. 31, 2018, which is a 35 USC 371 national stage filing of Patent Cooperation Treaty Application No. PCT/US2017/030922, entitled “Cardiac Mapping System with Efficiency Algorithm”, filed May 3, 2017, which claims priority to U.S. Patent Provisional Application Ser. No. 62/413,104, entitled “Cardiac Mapping System with Efficiency Algorithm”, filed Oct. 26, 2016, and U.S. Patent Provisional Application Ser. No. 62/331,364, entitled “Cardiac Mapping System with Efficiency Algorithm”, filed May 3, 2016, each of which is hereby incorporated by reference.

The present application, while not claiming priority to, may be related to U.S. patent application Ser. No. 16/961,809, entitled “System for Identifying Cardiac Conduction Patterns”, filed Jul. 13, 2020, which is a 35 USC 371 national stage filing of Patent Cooperation Treaty Application No. PCT/US2019/014498, entitled “System for Identifying Cardiac Conduction Patterns”, filed Jan. 22, 2019, which claims priority to U.S. Patent Provisional Application Ser. No. 62/619,897, entitled “System for Recognizing Cardiac Conduction Patterns”, filed Jan. 21, 2018, and U.S. Patent Provisional Application Ser. No. 62/668,647, entitled “System for Identifying Cardiac Conduction Patterns”, filed May 8, 2018, each of which is hereby incorporated by reference.

The present application, while not claiming priority to, may be related to U.S. patent application Ser. No. 17/048,151, entitled “Cardiac Information Processing System”, filed Oct. 16, 2020, which is a 35 USC 371 national stage filing of Patent Cooperation Treaty Application No. PCT/US2019/031131, entitled “Cardiac Information Processing System”, filed May 7, 2019, which claims priority to U.S. Provisional Application Ser. No. 62/668,659, entitled “Cardiac Information Processing System”, filed May 8, 2018, and U.S. Patent Provisional Application Ser. No. 62/811,735, entitled “Cardiac Information Processing System”, filed Feb. 28, 2019, each of which is hereby incorporated by reference.

The present application, while not claiming priority to, may be related to Patent Cooperation Treaty Application No. PCT/US2019/060433, entitled “Systems and Methods for Calculating Patient Information”, filed Nov. 8, 2019, which claims priority to U.S. Provisional Application Ser. No. 62/757,961, entitled “Systems and Methods for Calculating Patient Information”, filed Nov. 9, 2018, each of which is hereby incorporated by reference.

The present application, while not claiming priority to, may be related to U.S. patent application Ser. No. 17/601,661, entitled “System for Creating a Composite Map”, filed Oct. 5, 2021, which is a 35 USC 371 national stage filing of Patent Cooperation Treaty Application No. PCT/US2020/028779, entitled “System for Creating a Composite Map”, filed Apr. 17, 2020, which claims priority to U.S. Provisional Application Ser. No. 62/835,538, entitled “System for Creating a Composite Map”, filed Apr. 18, 2019, and U.S. Provisional Application Ser. No. 62/925,030, entitled “System for Creating a Composite Map”, filed Oct. 23, 2019, each of which is hereby incorporated by reference.

The present application, while not claiming priority to, may be related to U.S. patent application Ser. No. 17/613,249, entitled “Systems And Methods For Performing Localization Within A Body”, filed Nov. 22, 2021, which is a 35 USC 371 national stage filing of Patent Cooperation Treaty Application No. PCT/US2020/036110, entitled “Systems and Methods for Performing Localization Within a Body”, filed Jun. 4, 2020, which claims priority to U.S. Provisional Application Ser. No. 62/857,055, entitled “Systems and Methods for Performing Localization Within a Body”, filed Jun. 4, 2019, each of which is hereby incorporated by reference.

The present application, while not claiming priority to, may be related to U.S. patent application Ser. No. 17/777,104, entitled “Tissue Treatment Systems, Devices, and Methods”, filed May 16, 2022, which is a 35 USC 371 national stage filing of Patent Cooperation Treaty Application No. PCT/US2020/061458, entitled “Tissue Treatment Systems, Devices, and Methods”, filed Nov. 20, 2020, which claims priority to U.S. Provisional Application Ser. No. 62/939,412, entitled “Tissue Treatment Systems, Devices, and Methods”, filed Nov. 22, 2019, and U.S. Provisional Application Ser. No. 63/075,280, entitled “Tissue Treatment Systems, Devices, and Methods”, filed Sep. 7, 2020, each of which is hereby incorporated by reference.

### FIELD OF THE PRESENT INVENTIVE CONCEPTS

The present inventive concepts relate generally to systems, devices, and methods for ablating tissue, and in particular, for ablating tissue of a patient's heart.

### BACKGROUND

Numerous medical procedures include the mapping or other diagnosis of tissue, or the delivery of energy to ablate or otherwise treat tissue. Achieving desired specificity and efficacy of tissue diagnosis and treatment can be challenging, and the inability to do so can result in less than desired results.

There is a need for systems, methods, and devices that achieve improved tissue treatment via delivery of energy.

### SUMMARY

According to an aspect of the present inventive concepts, a cardiac information dynamic display system comprises one or more electrodes and a cardiac information console. The one or more electrodes are configured to record sets of electric potential data representing cardiac activity at a plurality of time intervals. The cardiac information console comprises a signal processor and a user interface module. The signal processor is configured to calculate sets of cardiac activity data at the plurality of time intervals using the recorded sets of electric potential data. The cardiac activity data is associated with surface locations of one or more cardiac chambers. The user interface module displays information related to the cardiac activity data. The information is presented relative to a graphical representation of surfaces of the one or more cardiac chambers.

According to another aspect of the present inventive concepts, a cardiac information dynamic display method, comprising using one or more electrodes, to record sets of electric potential data representing cardiac activity at a plurality of time intervals. The method also includes using a cardiac information console, comprising a signal processor and a user interface module: to calculate sets of cardiac activity data at the plurality of time intervals using the recorded sets of electric potential data, wherein the cardiac activity data is associated with surface locations of one or more cardiac chambers; and to display information related to the cardiac activity data, the information presented relative to a graphical representation of surfaces of the one or more cardiac chambers.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of an embodiment of a system configured to enable performance of a medical procedure on a patient, consistent with the present inventive concepts.

FIG. 2 illustrates an anatomic model representing a tissue surface, consistent with the present inventive concepts.

FIG. 3 illustrates an example of a graphical user interface displaying cardiac mapping data, consistent with the present inventive concepts.

FIGS. 4A and 4B illustrate an example of a graphical user interface including an anatomic model, and an anatomic model including various markers, respectively, consistent with the present inventive concepts.

FIGS. 5A and 5B illustrate a flow diagram representing an embodiment of a machine learning or other artificial intelligence-based method of rhythm classification and representations of cardiac activity data, respectively, consistent with the present inventive concepts.

FIG. 6 illustrates various embodiments of anatomic models upon which cardiac activity maps are displayed, consistent with the present inventive concepts.

FIG. 7 illustrates various embodiments of anatomic models upon which maps of cardiac activity are displayed, consistent with the present inventive concepts.

FIG. 8A illustrates an embodiment of a spatiotemporally-representative graph of cardiac activity, consistent with the present inventive concepts.

FIG. 8B illustrates an embodiment of a color-coded graph of cardiac activity, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” when used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component when used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” when used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, devices, and methods for treating target tissue of a patient, such as to provide a therapeutic benefit to the patient. An energy delivery console can be configured to deliver various “doses” of energy to be delivered by one or more energy delivery devices to ablate, cause the necrosis of, and/or otherwise therapeutically modify the target tissue. The one or more energy delivery devices can include catheters and/or surgical tools that include electrodes and/or other energy delivery elements. In some embodiments, multiple, inter-dependent energy doses are delivered to a common tissue location, such as to provide an improved therapeutic benefit to the patient. An initial dose can be configured to warm tissue, such as a delivery of radiofrequency (RF), heat, and/or other energy. A subsequent dose can comprise a dose of energy configured to irreversibly electroporate the tissue that had previously been warmed, such as while that tissue is in an elevated temperature (e.g. above body temperature) state.

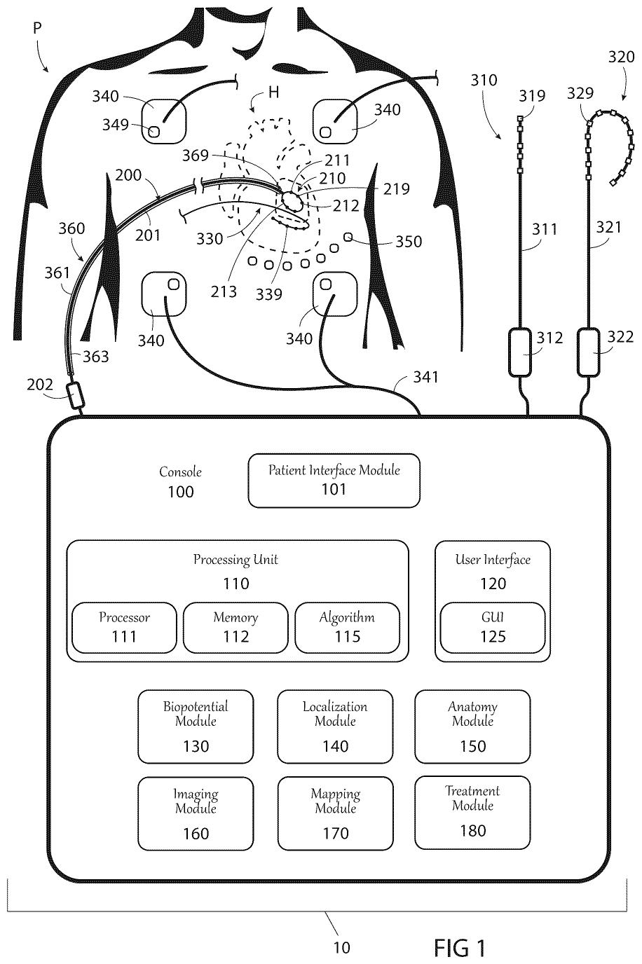

Referring now to FIG. 1, a schematic view of an embodiment of a system configured to enable performance of a medical procedure on a patient (e.g., a human or other living mammal) is illustrated, consistent with the present inventive concepts. The medical procedure can comprise a diagnostic procedure, a therapeutic procedure, or a combined diagnostic and therapeutic procedure that can be performed by a clinician and/or other user (“operator” or “user” herein). System 10 comprises console 100 that includes one or more discrete assemblies (e.g. individual boxes) that connect to various components of the system to provide energy thereto, record information from, and/or otherwise enable one or more functions of system 10 described herein. System 10 also includes one or more diagnostic catheters, mapping catheter 200 shown. In some embodiments, system 10 includes one or more treatment catheters, treatment catheter 310, one or more functional catheters, functional catheter 320, one or more additional diagnostic catheters, diagnostic catheter 330, one or more patient patches, patch 340, one or more patient leads, EKG lead 350, and/or one or more delivery devices, sheath 360. Console 100 operably attaches (e.g. electrically, mechanically, fluidly, sonically, and/or optically attaches) to the one or more catheters or other devices 200, 310, 320, 330, 340, 350, and/or 360.

Mapping catheter 200 can comprise an array of elements, array 210, handle 202, and an elongate filament therebetween, shaft 201. Array 210 can comprise a radially expandable array, such as an array resiliently biased in a radially expanded geometry. In some embodiments, array 210 can comprise a plurality of radially expandable arms, splines 213. Alternatively or additionally, array 210 can comprise a balloon, a radially expandable cage, or other expandable structure. Array 210 can comprise one or more functional elements, such as one or more electrodes, electrode 211, one or more ultrasound transducers, UST 212, and/or one or more other functional elements, functional element 219.

Mapping catheter 200 can be of similar construction and arrangement to the similar components described in applicant's co-pending: U.S. patent application Ser. No. 16/861,814, entitled “Catheter, System and Methods of Medical Uses of Same, including Diagnostic and Treatment Uses for the Heart”, filed Apr. 29, 2020; U.S. patent application Ser. No. 16/242,810, entitled “Expandable Catheter Assembly with Flexible Printed Circuit Board (PCB) Electrical Pathways”, filed Jan. 8, 2019; and U.S. patent application Ser. No. 17/735,285, entitled “Cardiac Virtualization Test Tank and Testing System and Method”, filed May 3, 2022.

Treatment catheter 310 can comprise an elongate filament, shaft 311, with handle 312 at its proximal end. Treatment catheter 310 can comprise one or more functional elements, functional element 319, positioned on a distal portion of shaft 311 (e.g. at least one functional element 319 positioned on the distal end of shaft 311). In some embodiments, functional element 319 comprises one or more electrodes configured to deliver electrical energy (e.g. RF energy) to tissue, such as to thermally ablate the tissue. Additionally or alternatively, functional element 319 can comprise one or more electrodes configured to generate an electrical field therebetween, such as to electroporate tissue within the field (e.g. to irreversibly electroporate the tissue).

Treatment catheter 310 can be of similar construction and arrangement to the similar components described in applicant's co-pending: to U.S. application Ser. No. 16/335,893, entitled “Ablation System with Force Control”, filed Mar. 22, 2019; U.S. application Ser. No. 17/777,104, entitled “Tissue Treatment Systems, Devices, and Methods”, filed May 16, 2022; and Patent Cooperation Treaty Application Serial No. PCT/US2022/016722, entitled “Energy Delivery Systems with Ablation Index”, filed Feb. 17, 2022.

Functional catheter 320 can comprise an elongate filament, shaft 321, with handle 322 at its proximal end. Functional catheter 320 can comprise one or more functional elements, functional element 329, positioned on a distal portion of shaft 321 (e.g. an array of at least 10 functional elements 329 positioned on the distal portion of shaft 321, such as 13 elements shown). In some embodiments, functional element 329 comprises one or more electrodes, such as one or more electrodes configured to record biopotential signals from cardiac tissue and/or other electrical signals.

Diagnostic catheter 330 can comprise a catheter including one or more functional elements, functional element 339. In some embodiments, diagnostic catheter 330 comprises a coronary sinus (CS) mapping catheter, which is constructed and arranged for positioning within the CS of the heart (e.g. to position functional element 339 within the CS). Functional element 339 can comprise one or more electrodes configured to record biopotential signals from cardiac tissue and/or other electrical signals.

Patch 340 can comprise one or more patches configured for application onto the skin of the patient (e.g. onto the torso of the patient). Patch 340 can comprise one or more functional elements, functional element 349. Functional element 349 can comprise an electrode configured to generate an electric field within the body of the patient (e.g. an electric field generated between at least two patches 340). System 10 can be configured to localize one or more devices thereof within and/or on the patient by measuring the electric field generated between patches 340 (e.g. via impedance-based localization described herein).

EKG lead 350 can comprise one or more patient patches configured to record electrical signals (e.g. cardiac electric signals) from the patient. Multiple EKG leads 350 can be positioned about the torso of the patient as shown.

Sheath 360 can comprise an elongate tube, shaft 361, including at least one lumen, lumen 363, therethrough. Sheath 360 can comprise at least one functional element, functional element 369 shown. Sheath 360 can be constructed and arranged to be intravascularly advanced into a chamber of the heart (e.g. into the left atrium of the heart via a transseptal puncture). Lumen 363 of sheath 360 can slidingly receive one or more devices of system 10, for example the distal portion of mapping catheter 200 (e.g. when array 210 is in a radially collapsed geometry), such that the device can be advanced from the distal end of lumen 363 and into a chamber of the heart. For example, array 210 of mapping catheter 200 can be advanced through lumen 363 of sheath 360 in a radially collapsed geometry, exit lumen 363 into the left atrium of the heart, and transition into a radially expanded geometry. In some embodiments, lumen 363 comprises two or more lumens configured to each slidingly receive a device of system 10, and/or lumen 363 is constructed and arranged to receive multiple devices simultaneously, such as to allow multiple devices (e.g. mapping catheter 200, treatment catheter 310, and/or functional catheter 320) to be inserted into the left atrium through a single transseptal puncture.

As used herein, devices 310, 320, 330, 340, 350, and/or 360 can be referred to singly or collectively as patient devices 300.

Console 100 can comprise patient interface module 101 configured to operably attach one or more patient devices (e.g. one or more catheters or other devices described herein) to one or more components of console 100. Patient interface module 101 can comprise circuitry configured to protect the patient, such as from undesired electric shock caused by console 100, and/or to protect the components of console 100 from electric shock, such as electric shock caused by defibrillation pulses or other energy delivered to the patient.

Console 100 can comprise processing unit 110. Processing unit 110 can comprise at least one microprocessor, computer, and/or another electronic controller, processor 111. Processing unit 110 can also include one, two, or more algorithms, algorithm 115 shown. Processing unit 110 can comprise memory 112 for storing instructions for performing algorithm 115. Processor 111, via algorithm 115, can perform one or more of the processes described herein, such as a process performed in response to one or more commands the user inputs into system 10 (e.g. via user interface 120 described herein). Processing unit 110 can receive a signal, such as a signal from one, two, or more functional elements of devices 200 and/or 300 (e.g. a signal from one, two, or more sensor-based functional elements of these devices). Processing unit 110 can be configured to perform one or more mathematical operations based on the received signal, and to produce a result correlating to a physiologic parameter of the patient and/or an operational parameter relating to at least one device of system 10.

Console 100 can include an interface for providing and/or receiving information to and/or from a user of system 10, user interface 120. User interface 120 can include one, two, or more user input and/or user output components. For example, user interface 120 can comprise a joystick, keyboard, mouse, microphone, touchscreen, and/or other input device. Additionally or alternatively, user interface 120 can comprise a speaker, haptic feedback device, indicator light, and/or other output device. In some embodiments, user interface 120 comprises one or more displays, such as a touch screen or other display for providing graphical visual information to the user. Processing unit 110 can provide a graphical user interface, GUI 125, to be presented to the user via user interface 120.

System 10 can comprise one or more modules for generating output signals (e.g. signals to be delivered to devices 200 and/or 300), receiving data (e.g. one or more recorded signals from devices 200 and/or 300), processing the received data (e.g. via algorithm 115), and/or generating output data based at least in part on the processed data. For example, system 10 can comprise biopotential module 130, localization module 140, anatomy module 150, imaging module 160, mapping module 170, and/or treatment module 180.

Biopotential module 130 can generate one or more outputs related to the electrical activity of the patient, for example dipole density information, surface charge information, and/or voltage information related to the activity of the patient's heart. Biopotential module 130 can be of similar construction and arrangement to similar components described in applicant's: U.S. Pat. No. 11,013,444, entitled “Method and Device for Determining and Presenting Surface Charge and Dipole Densities on Cardiac Walls”, filed Aug. 6, 2019; U.S. Pat. No. 11,116,438, entitled “Device and Method for the Geometric Determination of Electrical Dipole Densities on the Cardiac Wall”, filed Sep. 12, 2019; US Design Pat. No. D954,970, entitled “Set of Transducer-Electrode Pairs for a Catheter”, filed Feb. 28, 2019; and co-pending U.S. patent application Ser. No. 16/097,959, entitled “Cardiac Mapping System with Efficiency Algorithm”, filed Oct. 31, 2018.

Localization module 140 can generate one or more outputs related to the position of one or more components of system 10 relative to patient P, such as relative to a coordinate system established by localization module 140. Localization module 140 can be of similar construction and arrangement to similar components described in applicant's co-pending U.S. patent application Ser. No. 16/849,045, entitled “Localization System and Method Useful in the Acquisition and Analysis of Cardiac Information”, filed Apr. 15, 2020.

Anatomy module 150 can generate one or more outputs related to the anatomy of patient P, for example the size, shape, and/or structure of at least a portion (e.g. a chamber) of heart H of patient P. Anatomy module 150 can be of similar construction and arrangement to similar components described in applicant's: US Design Pat. No. D954970, entitled “Set of Transducer-Electrode Pairs for a Catheter”, filed Feb. 28, 2019; and co-pending U.S. patent application Ser. No. 17/735,285, entitled “Cardiac Virtualization Test Tank and Testing System and Method”, filed May 3, 2022.

Imaging module 160 can provide, produce, acquire, update, store, and maintain at least one image of the heart or at least one cardiac chamber of the heart H. Imaging module 160 can comprise at least one imaging device configured to record image data. For example, imaging module 160 can comprise an imaging device selected from the group consisting of: a computerized tomography (CT) scanner; a fluoroscope; an X-Ray imager; MRI scanner; an ultrasound imager; and combinations of these. In some embodiments, imaging module 160 receives and/or stores image information from the imaging device. Alternatively or additionally, imaging module 160 can be configured to receive image data from an imaging device separate from system 10. In some embodiments, imaging module 160 and anatomy module 150 are configured to provide, generate, and/or update an anatomic model, such as an anatomic model of at least a portion of the patient's heart, based on the image data from the imaging device.

Mapping module 170 can receive cardiac activity information (e.g. information recorded from devices 200 and/or 300 by biopotential module 130) and produce one or more maps of cardiac electrical activity. For example, mapping module 170 can produce one or more dipole density, surface charge, and/or voltage maps based on the recorded cardiac electrical activity. Mapping module 170 can be of similar construction and arrangement to similar components described in applicant's co-pending: U.S. patent application Ser. No. 17/673,995, entitled “Device and Method for the Geometric Determination of Electrical Dipole Densities on the Cardiac Wall”, filed Feb. 17, 2022; and U.S. application Ser. No. 16/097,955, entitled “Cardiac Information Dynamic Display System and Method”, filed Oct. 31, 2018.

Treatment module 180 can be configured to cause or drive a device of system 10 (e.g. treatment catheter 310) to deliver treatment energy to one or more locations of the patient's heart. In some embodiments, treatment module 180 provides closed-loop energy delivery based on mapping and/or other information produced by system 10. Treatment module 180 can be of similar construction and arrangement to similar components described in applicant's co-pending: U.S. patent application Ser. No. 16/861,814, entitled “Catheter, System and Methods of Medical Uses of Same, including Diagnostic and Treatment Uses for the Heart”, filed Apr. 29, 2020; U.S. application Ser. No. 16/335,893, entitled “Ablation System with Force Control”, filed Mar. 22, 2019; and U.S. patent application Ser. No. 17/777,104, entitled “Tissue Treatment Systems, Devices, and Methods”, filed May 16, 2022.

In some embodiments, system 10 is configured to produce one or more maps of cardiac activity (e.g. via mapping module 170) based on information gathered in a non-contact manner (e.g. recorded from one or more electrodes positioned within a cardiac chamber without contacting the cardiac wall, “non-contact data” herein). Alternatively or additionally, system 10 can be configured to produce one or more maps of cardiac activity based on information gathered in a contact manner (e.g. recorded from one or more electrodes positioned in contact with the cardiac wall, “contact data” herein). In some embodiments, system 10 is configured to generate one or more “hybrid” maps of cardiac activity based on both contact data and non-contact data.

Localization module 140 can provide signals to, and/or record signals from, one or more devices of system 10. For example, localization module 140 can provide one or more signals to patches 340, such as to establish an electric field within the patient. Localization module 140 can record signals from one or more devices of system 10 relating to the established electric field to determine the location and/or orientation of that device within the field via an impedance-based method (to “localize” the device). Alternatively or additionally, localization module 140 can localize one or more devices of system 10 via a magnetic-based method, such as when localization module 140 is constructed and arranged to establish a magnetic field within at least a portion of the patient (e.g. via one or more permanent magnets and/or electromagnets), and one or more functional elements of system 10 comprise magnetic coils or other elements configured to detect magnetic fields.

In some embodiments, localization module 140 is configured to localize one or more devices of system 10 that have been placed in a static location with minimal further intentional movement expected (e.g. movement by an operator), for example diagnostic catheter 330 when placed within the CS. Localization module 140 can be configured to track the location of this static device, and to utilize this device as a “physical reference” (e.g. a localizable physical point within the patient that is unlikely to move relative to the heart, as described herebelow). In some embodiments, localization module 140 can localize one or more other devices of system 10 (e.g. any or all other devices that are present within the patient, such as positions within or proximate the heart), based at least in part on the relative position of the physical reference to the additional devices being localized.

System 10 can be configured to navigate (e.g. provide navigation information and/or automatically navigate) one or more catheters and/or other devices of system 10. System 10 can perform navigation of devices using impedance measurements recorded by system 10 (e.g. by localization module 140), as described herein. System 10 can perform impedance measurement in a number of different ways. In some embodiments, system 10 uses a set of multiple (e.g. at least three) pairs of patches (e.g. three pairs of patches 340) to deliver electrical current through the body. Three patch pairs can deliver unique frequencies (e.g. simultaneously). Sensors (e.g. electrodes) within the body can measure the potential at each of the three unique localization frequencies relative to a reference measurement made elsewhere on the body (such as a patch on the body surface low on the torso).

System 10 can be configured to perform impedance-based device navigation while reducing effects of motion artifacts and/or other artifacts, such as through the use of “adaptive referencing”. In some embodiments, system 10 tracks a set of designated electrodes (e.g. from one or more catheters and/or other devices of system 10) that are likely (e.g. highly likely) to be placed in a “static location”, such as when positioned in a location in which minimal further movements are expected (e.g. minimal manipulations by an operator are expected, such as when placed in the coronary sinus or other intracardiac location in which further manipulation is not needed and/or desired). System 10 can be configured to keep track of those electrode locations, and use them as a “physical reference”. For example, system 10 can track other devices (e.g. catheters that are to be manipulated) by using the physical reference as an “anchor”, such as by subtracting the real-time location of the physical reference. System 10 can be configured to compensate for physical displacement of any electrodes that are being used as physical references, where such displacements cause a shift of the coordinate system. In some embodiments, system 10 uses a dynamic, adaptive learning model to train a set of measurements from body surface sensors (e.g. patches 340) to create a set of virtual intracardiac measurements that quantitatively reconstruct a signal equivalent to a physical device, a “virtual reference” herein. For example, system 10 can track other devices (e.g. catheters) by using the virtual reference as an anchor, such as by subtracting the real-time location of the virtual reference. These virtual references of system 10 are not susceptible to the types of physical motion that can be encountered when using a device inserted into the patient (e.g. an intracardiac catheter as described hereabove), but they are susceptible to disturbances associated with the body surface sensor itself, the localization measurement reference (e.g. a reference patch electrode), and/or electrical disturbances introduced to (e.g. associated with) a single device (e.g. single catheter), multiple devices (e.g. multiple catheters), and/or the entire system. In some embodiments, system 10 can be configured to store (e.g. in memory) a time-history of all tracked locations (e.g. device locations, physical reference locations, and/or virtual reference locations), such as to recall (e.g. and use) previous locations of any device (e.g. to be used in a localization and/or localization compensation of one or more system 10 devices). This stored information can be useful in detecting differences between systemic disturbances versus disturbances specific to a single device (e.g. a single catheter). For example, electrical disturbances can affect a single device, multiple devices, and/or the entire system, and device motion will be unique to each individual device. Each of these above methods can be calculated simultaneously and/or sequentially.

In some embodiments, system 10 is configured to perform an impedance-based device (e.g. catheter) navigation method using a “position agreement algorithm” (e.g. an algorithm of algorithm 115) that is configured to account for motion artifacts and/or other measurement artifacts. If system 10 utilizes more than one device tracking method (e.g. two or more of those described hereabove), system 10 can implement a position agreement algorithm to cross-compare the solutions of each of the multiple methods to determine one or more subsets of solutions that are “in agreement”. Alternatively or additionally, the system 10 position agreement algorithm can determine one or more subsets of solutions that are indicative of an aberration. The position agreement algorithm can bias system 10 to prioritize one or more of these methods, and/or to de-prioritize (e.g. ignore) one or more of these methods, based on determining agreement and/or finding indications of an aberration. In some embodiments, prioritization and/or de-prioritization can be performed by assigning a weighting factor (e.g. a quantitative weighting factor) to each of these localization methods. In some embodiments, the position agreement algorithm can designate when a de-prioritized method (e.g. a method that is currently not being used) is to be reinstated (e.g. used again). The position agreement algorithm can use a set of predefined logic rules to determine the possible root cause of disturbance, such as to make appropriate adjustments to mitigate. If the physical reference, virtual reference, and time-history of locations all fall within a designated distance (e.g. like a geo-fence), system 10 can determine that all tracking methods are in agreement. However, if the physical reference and the virtual reference agree, but the most recent stored location(s) in the time-history does not agree, then an abrupt electrical disturbance is likely affecting the location of at least the designated reference device. Comparing the locations of other devices (e.g. catheters) in the body to their time-history positions can help differentiate catheter-specific versus full system disturbances. The overall quantitative effect of the disturbance on location can be mitigated by correcting the tracked position of the physical reference and virtual reference to align with the most recent valid time-history location. For example, if the physical reference and the time-history of the physical reference agree, but the virtual reference disagrees, the virtual reference may be aberrant and can be ignored by system 10 (e.g. until it comes back into agreement with the physical reference and time-history of the physical reference, after which it can be reinstated).

In some embodiments, system 10 is configured to perform an impedance-based device (e.g. catheter) navigation method using a “position determination algorithm” (e.g. an algorithm of algorithm 115), such as an algorithm configured to utilize the results of the tracking methods and position agreement algorithm described hereabove to determine (e.g. and display) the position of one or more (e.g. all) of the system 10 devices being used (e.g. position of one or more electrode of one or more catheters and/or other devices positioned in and/or on the patient). In some embodiments, the position determination algorithm is configured to produce a “probability score” comprising a confidence, and/or other probability measurement (e.g. a quantitative output) regarding the determined position of one or more devices. The probability score can be displayed to the user via an alphanumeric value that is displayed, and/or by modifying (e.g. augmenting) the way the displayed device is shown (e.g. hash marks, color changes, brightness changes, and/or other visual differentiation of the body and/or another portion of the associated catheter and/or other device). In some embodiments, system 10 comprises various thresholds (e.g. quantitative thresholds) that are used by algorithm 115 to classify a device's predicted location by comparing the probability score to one or more thresholds.

In some embodiments, system 10 comprises a “data quality routine” configured to assess the quality of data from one or more connected measurement devices of system 10. The data quality routine can be configured to determine a “status” of a system 10 device, such as to determine whether a system 10 device is electrically disconnected, has been removed from the body, is not deployed (e.g. not fully deployed), and/or has sensors that are degraded. In some embodiments, the data quality routine of system 10 (e.g. as implemented by algorithm 115) determines the status of the device by comparing a measured and/or calculated parameter associated with the status (e.g. as determined by a sensor of system 10) to a threshold of system 10 associated with the status.

In some embodiments, system 10 (e.g. algorithm 115) is configured to perform “dynamically bounded adaptive impedance tracking” with a monitored localization reference, such as using one or more of the methods described hereabove (e.g. using one or more electrodes to calculate a virtual reference). In some embodiments, system 10 uses one or more sensors (e.g. electrodes) that are positioned in the body and designated as a monitored localization reference. The physical device can be expected to be left in a static location for a time period (e.g. a time period above a minimum amount of time). System 10 can monitor this device to determine if the device has been physically displaced, and/or if an electrical disturbance has affected the device (e.g. the device alone), one or more other devices (e.g. multiple devices), or the entire system. System 10 can be configured to determine if any additional devices other than the monitored localization reference are positioned (e.g. currently located) within the body. When more than one device is available in the body, system 10 can determine whether any of the devices are moving due to user manipulation and/or are independently being affected by electrical disturbance. System 10 can be configured to keep track of historical locations of the monitored localization reference (e.g. and other devices), such as when electrical disturbances have not occurred. System 10 can create a set of virtual measurements (e.g. virtual intracardiac measurements) that quantitatively reconstruct a signal equivalent to a monitored localization reference. System 10 can use a full set of body surface measurements (for example, a functional element of system 10 comprising a 12-lead ECG) to construct this virtual reference. System 10 can (e.g. alternatively) use a set of body surface pairs (fewer than the full set) to construct the virtual reference, where the set of body surface pairs are selected to most closely bound (quantitatively) the location data of the monitored localization reference device. This approach can be more accurate and resilient to large disturbances to the location of catheters. System 10 can track the monitored localization reference device, the virtual reference, and/or all other devices positioned (e.g. at least a portion is positioned) in the coordinate system being used by system 10. System 10 can adaptively mitigate physical and electrical disturbances. For example, when the monitored localization reference device is physically displaced, the virtual reference can be recomputed to match, and the time-history data can receive a valid, updated location of the monitored localization reference device. Alternatively or additionally, when electrical disturbances affect the virtual reference and/or the tracked location of the monitored localization reference, the virtual reference can be recomputed (e.g. by algorithm 115), and the location displacement caused by the disturbance can be mitigated by computationally realigning the tracked location of the monitored localization reference to the last known good position in the time-history.

In some embodiments, system 10 is configured to perform an impedance-based device (e.g. catheter) navigation method in which compensation is performed to account for patient respiration. As described herein, system 10 can use electrodes of devices that are in a static location to create a “physical respiratory reference”. System 10 can be configured to filter motion of the associated device to a range of frequencies related to respiration, such as frequencies less than or equal to 1.0 Hz, 0.5 Hz, and/or 0.3 Hz. System 10 can use this measured respiratory signal to compensate for respiratory motion, such as by subtracting it from the raw localization signal on any electrode. Alternatively or additionally, system 10 can use one or both of a “gating method” and/or “dynamic learning model” to account for patient respiration. For example, system 10 can use a gating method that establishes a consistent period of respiratory cycle, using measurements from the body surface, from within the patient's body, or both. The gating method can simplify data collection by: only using data acquired during the gated range of the respiratory cycle, such as for discrete acquisition similar to collecting contact mapping points (e.g. anatomic “shell” and/or EGM); and/or performing a sample and hold for device positions from one gated period to the next for time-continuous measurements (e.g. while employing interpolation between gated positions). System 10 can use a dynamic learning model (e.g. an adaptive model) to train a set of body surface measurements (e.g. via patches 340) to create a set of virtual intrabody (e.g. intracardiac) respiration measurements that retain the respiratory component of motion (e.g. using filtering as a technique to keep only the respiratory component). System 10 can use this measured respiratory signal to compensate for respiratory motion, such as by subtracting it from the raw localization signal on any electrode. System 10 can be configured to perform two or more of the above respiration compensation methods simultaneously and/or sequentially. In some embodiments, system 10 performs multiple respiration compensation methods (e.g. as described herein), where each method is assigned a weighting factor used by system 10 to prioritize and/or otherwise apply different levels of importance and/or impact (“prioritize” herein) on one method versus another.

In some embodiments, system 10 is configured to track, monitor, analyze, and/or compensate for patient respiration using a “respiration tracking agreement algorithm” (e.g. an algorithm of algorithm 115). If more than one respiration compensation method described hereabove is employed by system 10, a respiration tracking agreement algorithm can be used by system 10 to cross-compare the solutions of each method to determine the one or more subsets of solutions that are in agreement, and/or to determine the one or more subsets of solutions that are indicative of an aberration. This respiration tracking agreement algorithm can designate the methods to temporarily “ignore” and also determine when an “ignored” method should be included again, as described herein.

In some embodiments, system 10 is configured to compensate for patient respiration using a “respiration compensation algorithm” (e.g. an algorithm of algorithm 115), such as an algorithm configured to utilize the results of the respiration compensation methods and respiration tracking agreement algorithm described hereabove to determine (e.g. and display) the respiratory-compensated position of one or more (e.g. all) of the system 10 devices being used (e.g. position of one or more electrodes of one or more catheters and/or other devices positioned in and/or on the patient). System 10 can perform the compensation using direct subtraction of the tracked respiratory motion from all associated device (e.g. electrode) positions. System 10 can perform the compensation by keeping track of respiratory motion differently in different locations of the anatomy, and by subtracting the local respiratory motion from displayed device (e.g. electrode) positions in the associated location. The different anatomical locations can be organized on a regular spatial grid (e.g. using voxels). In some embodiments, the respiration compensation algorithm is configured to produce a “probability score” comprising a confidence, and/or other probability measurement (e.g. a quantitative score) regarding the compensation applied in determining the determined position of one or more devices (e.g. the position determined using respiratory compensation). For example, the probability score can be based on the probability that a disturbance is encountered, and/or it can be based on the degree of residual respiratory motion that remains uncompensated. The probability score can be displayed to the user via an alphanumeric value that is displayed, and/or by modifying (e.g. augmenting) the way the displayed device is shown (e.g. hash marks, color changes, brightness changes, and/or other visual differentiation of the body and/or another portion of the associated catheter and/or other device). In some embodiments, a respiration compensation algorithm of system 10 (e.g. as implemented by algorithm 115) compares the probability score to a threshold of system 10 associated with the respiration compensation algorithm (e.g. to classify the compensation and/or to modify the way a displayed device is shown).

In some embodiments, system 10 is configured to compensate for patient respiration using an “impedance navigation accuracy optimization and scaling algorithm” (e.g. an algorithm of algorithm 115). Variations in the impedance of body structures and composition can affect the accuracy of navigation performed by system 10. These variations can be computationally compensated by keeping track of their effect on measured distances at different locations in the body. The process performed by system 10 of estimating the relationship at a given location between a set of impedance measurements and a set of corresponding known distances (for example physical spacings between electrodes) can be referred to as “scaling”. By tracking these variations and computationally adjusting for them with variable scaling at different locations in the body, the impedance navigation becomes more accurate. Initially, when device (e.g. catheter) measurements are first made in the body, there is limited impedance data available. The limited data can be used to initially estimate scaling in the body. As devices are maneuvered throughout the body, the scaling information becomes more discretely measured and scaling information becomes more granularly refined. This system 10 process is referred to as “dynamic scaling”. The impedance navigation accuracy optimization and scaling algorithm can comprise an “impedance scaling optimization algorithm” (e.g. an algorithm of algorithm 115) that processes the set of discrete scaling measurements to create a cohesive coordinate space where measured impedance data can be directly mapped to unique coordinate locations. As more measurements are made, the impedance scaling optimization algorithm can be iteratively executed (for example on a 1 sec or 5 sec interval) to provide increasingly accurate navigation of devices. As navigation accuracy is improved, previously collected location information can be retrospectively updated. Any data stored and/or calculated by system 10 that is determined using location information can be accordingly recalculated (e.g. “refactored”). This data includes anatomy measurements, electrical measurements, and/or marked locations from device locations. Some information not directly derived from device locations can also be recalculated by system 10. For example, marked locations placed in the coordinate space that were not made from a measured device location (for example, placing a marker on the anatomy with a mouse) can be saved with corresponding “equivalent impedance” based on the scaling information available at that time. When the location data is recalculated, the location of these markers can be recomputed using the equivalent impedance, and it can be displayed (e.g. in a new location on the screen).

In some embodiments, system 10 comprises one or more components configured to provide magnetic data (e.g. data recorded by and/or derived from magnetic components), such as to be used in a hybrid localization system. System 10 can utilize a magnetic navigation system to simultaneously track devices in the body using a second (e.g. different) measurement modality (e.g. different than those described hereabove). System 10 (e.g. algorithm 115) can be configured to perform hybrid mitigation of disturbances. System 10 can utilize the redundancy of measurements to mitigate disturbances to an impedance tracking subsystem and/or disturbances to a magnetic tracking subsystem. Monitoring algorithms (e.g. one or more algorithms of algorithm 115) can be used for each modality to determine (e.g. repeatedly and/or relatively continuously determine) if disturbances affecting either modality are occurring. When disturbances are detected by algorithm 115, the affected subsystem can be temporarily disengaged from affecting the display of tracked devices (e.g. catheters) until the system determines that the associated disturbance has been mitigated and/or is no longer occurring. System 10 can be configured to perform hybrid navigation accuracy optimization and scaling. When used in conjunction with an impedance navigation subsystem, the magnetic subsystem's accuracy is less impacted by variations of the body structures and composition. The magnetic navigation subsystem can therefore provide (e.g. more immediately) accurate navigation data that can be used to construct the impedance scaling data (e.g. more rapidly) as devices are maneuvered throughout the body. System 10 can utilize the magnetic data as a computational backbone for the impedance scaling optimization, replacing the use of “known distances” (e.g. the physical spacing between electrodes) to indirectly map out the impedance variations with a directly measured correspondence between accurate locations in space and impedance measurements. System 10 can navigate devices that include only impedance sensors, only magnetic sensors, or both. Devices equipped with both types of sensors can be used to directly construct the impedance correspondence map into the displayed coordinate system. Impedance scaling optimization may not be necessary in areas where both magnetic data and impedance data have been directly measured.

System 10 can be configured to “reconstruct” and store a portion of the patient's anatomy, such as to provide a reconstruction of one or more portions of the patient's heart. The stored anatomy can be used as an input to: data calculation algorithms (e.g. one or more algorithms of algorithm 115 configured to perform inverse solution calculations); processing & display algorithms (e.g. one or more algorithms of algorithm 115 configured to calculate and/or otherwise determine contact point acceptance criteria, closest surface location and/or direction, and/or an estimation of therapy delivery into tissue); and/or as a visual “canvas” onto which many forms of data can be displayed. The anatomy information can comprise one or more anatomical components, such as distinct anatomic structures, such as to differentiate different chambers of the heart, veins, arteries, and/or appendages. The anatomy information stored can comprise point location data, surface (e.g. a shell) data, volume data, data from direct measurements of anatomy properties (e.g. density, thickness, tissue type, tissue composition), data from calculations and/or estimations of anatomy properties (e.g. normal direction from a surface, angle of incidence to an object, conduction properties such as fiber orientation, scar heterogeneity, preferential pathways or connections to other structures, and the like). System 10 can be configured to record, store, process, and/or display image data to collect the anatomy information. The anatomy information stored can comprise data recorded by system 10, and/or data that is provided to system 10. In some embodiments, the image data is determined by locating objects in the body. The location of objects can be determined using one or more points at a time by determining imaged points, which can be processed to form an imaged surface and/or an imaged volume.

In some embodiments, system 10 includes one or more devices (e.g. catheters and/or external devices) that transmit and/or receive ultrasound signals, such that the resultant ultrasound data can be converted into image data, such as an anatomic “shell” of the patient's heart wall tissue and/or other tissue of the patient (e.g. non-blood tissue). Imaged points can be created from ultrasound reflection data. In some embodiments, the ultrasound data comprises reflection data from one or more transducers. The location of a point from which ultrasound is reflected can be used to determine the location of an object within the heart (an “imaged point”), such as a cardiac wall. System 10 can be configured to determine the point from which ultrasound is reflected by determining three fundamental elements: a point of origin (transducer location), direction of transmission & detection (vector), and a range to target.

System 10 can be configured to update and/or recalculate (e.g. refactor) one or more imaged data points. System 10 can keep track of each imaged point (the calculated ultrasound point) and its three fundamental elements. In some embodiments, system 10 can determine updated information (such as location information with greater accuracy) which can retrospectively be used to modify the fundamental elements used to calculate each imaged point. System 10 can then recalculate updated imaged points from the updated fundamental elements. For example, if one or more device navigation algorithms of system 10 (e.g. algorithm 115) provide updated device locations, the point of origin and/or the direction of transmission and detection for each imaged point can be accordingly updated. System 10 can then recalculate the anatomy surface from the updated imaged points. Subsequent calculations made by system 10, such as are described herein, can be updated based on the new anatomy surface and/or based on imaged point information.

System 10 can be configured to created imaged points from various forms of image data. System 10 can utilize various image data from ultrasound imaging devices (e.g. B-mode ultrasound imaging devices), CT scanners, X-ray imagers, and/or MRI imaging devices to determine and integrate imaged points. Such image data includes data where objects can be differentiated based on intensity, color, brightness and/or other quantitative values in a 2D plane or 3D volume. This image data can be converted to imaged points by determining the orientation and registration of the image data within the coordinate space tracked by system 10. System 10 can select the one or more ranges of values within the image data that represent objects of interest, determine the corresponding imaged points in the system's coordinate space, and integrate the new imaged points. In some embodiments, system 10 utilizes data from two or more of the imaging devices described herein. In these embodiments, a weighting factor can be applied to apply different levels of importance and/or impact to data obtained from one imaging device versus another imaging device.

System 10 can be configured to organize imaged points into a data structure. System 10 can keep track of location information in a coordinate space, for instance a three-dimensional rectilinear and/or Cartesian coordinate system. Within the coordinate space, system 10 can organize the imaged point data into a data structure, such as a 3D voxel space, wherein, for example, each voxel designates a unique range of volumetric space in the coordinate system and each voxel can contain: no imaged points, a single imaged point, or multiple imaged points. The voxels can be uniform in each direction (such as when part of a volume of 0.5 mm by 0.5 mm by 0.5 mm, and/or a volume of 1 mm by 1 mm by 1 mm). The voxels can be consistent in size and/or the voxels can vary in size. The voxels can be adaptively merged and/or divided to efficiently organize and process the contained data. The merging and/or dividing can be based on the contained data within the voxels, such as the location of data within each voxel and/or the number or density of data within each voxel. The data structure created by system 10 can comprise an octree data structure and/or other highly efficient data architecture, such as to enable efficient data search and/or data processing algorithms (e.g. one or more algorithms of algorithm 115). Additionally or alternatively, the data structure can take the form of a structured computational mesh (e.g. a rectilinear grid) and/or an unstructured computational mesh (e.g. a tetrahedral mesh), for example when the manifold-neighborhood is explicit in the mesh definition used by system 10 and/or must be determined by system 10 after creation of the mesh.

System 10 can be configured to determine attributes and/or quantitative metrics that can be used to analyze imaged points. The data structure created by system 10 can keep track of attributes and/or quantitative metrics of each voxel, such as to enable processing efficiency. In some embodiments, each voxel has an attribute related to (e.g. keeps track of the attribute related to): being filled or empty; the number of imaged points within the voxel; and/or the density of points within the voxel. Each voxel can keep track of the geometric center of the voxel. Each voxel can keep track of the geometric centroid of the contained points within the voxel. As imaged data is added, removed, and/or changed, the attributes and/or quantitative metrics can be accordingly updated by system 10. System 10 can keep track of multiple parallel data structures such that points can be designated to different data structures to keep track of independent point sets. For example, system 10 can allocate points designated to be from a left chamber of the heart to a first data structure and points designated to be from a right chamber of the heart to a second data structure. Alternatively, system 10 can use a single data structure to organize all points and use attributes of the voxels and/or the individual points to keep track of such designations.

System 10 can be configured to identify artifactual imaged points. Imaged points may artifactually appear within the interior of an anatomic structure. In some embodiments, imaged points that are located within a threshold distance of any previous locations of intracardiac devices may be excluded from further calculations. In some embodiments, system 10 comprises multiple threshold distances that are used to exclude one or more devices (e.g. multiple thresholds correlating to different device types or other differentiation).

System 10 can be configured to generate a surface (e.g. a shell or a part of a shell), such as a surface representing a cardiac wall and/or other tissue surface of a patient. System 10 can create a surface from a set of imaged points. In some embodiments, the surface is computed from a set of points using a Poisson surface. In some embodiments, a data structure of voxels is used by system 10 to organize a set of imaged points. Voxels that are “filled” are used to generate a surface. Organizing the imaged points into voxels can improve the efficiency of the surface calculation, as the data density of the raw set of imaged points can far exceed the necessary resolution required to create a surface. In some embodiments, the locations of the filled voxels (e.g. the center point of each voxel) are used by system 10 (e.g. an algorithm 115 of system 10) as surface guidance points. Each surface guidance point is associated with a vector whose direction is estimated to be in the normal direction of the desired calculated surface. In some embodiments, system 10 approximates the normal vector using the normalized radial projection from a central point in the coordinate system to each voxel's center. The central point is preferred to be within and near the center of the anticipated closed surface. In anatomic applications, choosing a point central to the anatomic structure is appropriate. The central point can be reassigned by system 10 as desired and/or necessary. In some embodiments a surface can be calculated by system 10 by first establishing a set of Gaussian basis functions, the locations of which are placed approximately at each surface guidance point. From the Gaussian basis functions, along with the normal vectors, a solution of the Poisson equation (below) defining the surface is obtained:

$$∇ \cdot ∇ ϕ = ∇ \cdot \vec{n}$$

The solution ϕ of the governing equation is obtained discretely by an inverse method which yields the “strength” of each Gaussian basis function. Briefly, one realization of the governing equation is written for each surface guidance point, and the divergence of the normal at that point is expressed as a linear sum of contributions from nearby surface guidance points. To limit the region of influence around each surface guidance point, the basis functions are defined with compact support. This yields a sparse influence matrix which facilitates rapid solution. The calculated surface is chosen as the closed iso-surface of value ϕ=0.5.

The surface can be represented as a triangulated mesh. The resulting surface will be an enclosed surface following the guidance points as closely as possible. In areas where there are no guidance points or limited guidance points, the calculated surface may be poorer in accuracy and can be removed from the triangulated mesh. In some embodiments, system 10 does not calculate a surface until a sufficient number (e.g. 48, 64 or 100) of guidance points distributed in the coordinate system is collected. In some embodiments, a “sufficiency algorithm” of system 10 (e.g. one or more algorithms of algorithm 115) can determine when the number and distribution of guidance points is sufficient to initiate calculation of the surface (e.g. when a threshold is exceeded). In some embodiments, the calculated surface is biased toward the interior or exterior of the set of surface guidance points. A “scaling algorithm” of system 10 (e.g. one or more algorithms of algorithm 115) can be applied that estimates the signed offset of each surface guidance point to the calculated surface, and repositions each vertex of the calculated surface such that the mean offset vanishes. In some embodiments, the surface calculation process of system 10 generates isolated segments of the surface, and an algorithm 115 comprising an isolated segment removal algorithm eliminates these structures.

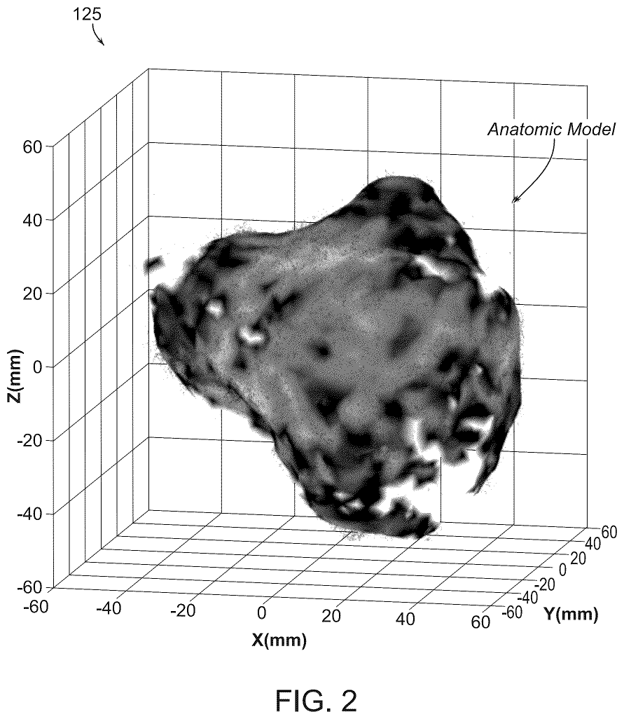

In some embodiments, system 10 can create a visualization of a surface from imaged points, for example as described herein in reference to FIG. 2. System 10 can be configured to iteratively calculate and display the imaged surface as imaged point data is continuously collected. System 10 can display raw imaged points, surface guidance points, the surface mesh, or any combination of these. In some embodiments, the surface mesh can be displayed as a single color. In some embodiments, the surface mesh can be differentially visualized (e.g. via variations in color, transparency, intensity, and the like) based on attributes and/or quantitative data of the nearby surface guidance points. For example, the surface can be colored and/or otherwise graphically differentiated based on the density of imaged points within the nearby voxels. This visual differentiation can allow the user to observe areas where sufficient data has been collected and/or where data collection has been limited such that the user can adjust the data collection accordingly. The opacity of portions (e.g. triangles) of the anatomic model mesh can also be based on the density of imaged points within the nearby voxels. During live collection of data and/or iterative calculation of the surface, the visualized surface can have an initial appearance to facilitate the user's interpretation of the data collection process and/or to optimize calculation efficiency and processing speed. When not actively collecting, the visualized surface provided by system 10 can have an optimized appearance. In some embodiments, the recalculation and update to the display by system 10 reinitiates at regular intervals, such as intervals of no more than 0.5 seconds, 1 second, and/or 2 seconds. In some embodiments, the recalculations and update to the display reinitiate asynchronously with the collection of additional imaged points. In some embodiments, an “update reinitiation algorithm” of system 10 (e.g. one or more algorithms of algorithm 115) is used to estimate and/or calculate the degree of change that newly acquired data introduces, and the algorithm can determine when the acquisition of new data surpasses a threshold that initiates a recalculation of the surface and corresponding update to the display. In some embodiments, system 10 uses a combination of reinitiation methods (e.g. where each reinitiation method is assigned a weighting factor, such as to apply a differentiating importance to each method). In some embodiments, system 10 uses the shorter interval of any one of a combination of reinitiation methods.

System 10 can be configured to create an anatomic reconstruction comprising a volume reconstruction. In some applications, the anatomy can be used as a volume structure rather than a surface structure. If a surface structure has already been created, system 10 can calculate a volume structure by filling the internal volume of the surface with points and generating a tetrahedral volume mesh to represent the volume object. In some embodiments, the set of internal points can be determined (e.g. rigorously determined) by testing each point on a regular 3D grid to determine if it falls inside or outside of the surface. In some embodiments, an “internal point placement algorithm” of system 10 (e.g. one or more algorithms of algorithm 115) can efficiently search for points that are internal to the surface and can determine a set of internal points at a density sufficient to create a tetrahedral volume mesh. In some embodiments, the internal point placement algorithm can use a first seeded point internal to the enclosed boundary of the surface, construct large tetrahedral segments with portions of the surface (e.g. of uniform or otherwise similar size), and iteratively subdivide the tetrahedra into smaller and smaller sizes until a threshold (such as desired number of internal points, an internal point density, a mean tetrahedral edge length or volume) is met. Alternatively, the internal point placement algorithm can perform a search using compartmental subdivision to test points on a regular 3D grid, where the data space is divided into large compartments, and each boundary point of the compartments are tested to be classified as being inside or outside of the surface. Compartments where one or more boundary points are internal are subdivided, and the process is repeated for each subdivision of the previous compartment. In some embodiments, boundary points previously tested are not retested. This subdivision process can proceed until a threshold (such as desired number of internal points, an internal point density, and/or a mean tetrahedral edge length or volume) is met. In some embodiments, system 10 comprises multiple of these thresholds, which can be used to categorize (e.g. quantify or qualify) the quality (e.g. the resolution, accuracy, and the like) of the output of the process.

In some embodiments, multiple tetrahedral meshes (e.g. low and/or high resolution meshes) are pre-computed, and upon completion of the anatomical shell by system 10, a bounding box of the mesh can be estimated (e.g. by algorithm 115 of system 10), and the pre-computed mesh can be transformed to a bounding box of the anatomy. In some embodiments, following the transformation of the pre-computed mesh, each node of the mesh can be evaluated (e.g. by algorithm 115 of system 10) and categorized as falling inside or outside the anatomical mesh.

In some embodiments, the pre-computed mesh comprises varying resolution with dense tetrahedra near the center of the bounding box and sparse tetrahedra towards the outside of the bounding box. The parameterization of this resolution can be determined (e.g. by algorithm 115 of system 10) based on population level average shapes and metrics computed from them, such as distance functions.

System 10 can be configured to create an anatomic reconstruction by creating anatomy data from tracing catheter and/or other device locations. System 10 can create anatomy data by tracking the location of navigated devices and determining the outer boundary of the volume “traced out” by the device's various positions. The tracked positions of devices, in aggregate, can by used by system 10 (e.g. algorithm 115) to form a representation of an anatomical volume.

System 10 can be configured to create anatomy data by integrating anatomy data collected from multiple methods (e.g. two or more of the methods described herein or otherwise). In some embodiments, system 10 integrates various (e.g. all or a portion of) anatomy data collected (e.g. by system 10) using multiple methods. In some embodiments, imaged surface data is converted to volume data by system 10. Device-traced data can also be represented as volume data (e.g. volume data comprising structured and/or unstructured elements). Integration of the two data sets can be performed (e.g. by algorithm 115) by merging the volume data in the same coordinate space, and processing the union of both data sets as a cohesive volume.

System 10 can be configured to edit (e.g. allow editing by an operator and/or automatically edit) anatomy structures. System 10 can be configured to enable the user to add, remove, and/or edit anatomy structures. In some embodiments, surface or volume data can be deleted and/or modified (e.g. “shaved”). Surface or volume data can be re-assigned to different structures. Surface data can be modified by cutting a hole in a surface mesh. In some embodiments, a user selectable geometric shape (e.g. a predefined geometric shape stored in a library of system 10) is used to facilitate the creation of the anatomy. For example, one, two, three, or more of each of spherical, elliptical, teardrop, and/or other shapes can be provided by system 10 and used to facilitate the initial creation of the anatomy.

System 10 (e.g. via algorithm 115) can be configured to perform cardiac signal (e.g. EGM) optimization. For example, system 10 can be configured to perform far-field compensation. In some applications, such as by inversely-solving for atrial activation, the far-field influence of activation in a chamber other than the atria being diagnosed and/or treated, such as a ventricle or the opposite atria, can be disruptive. It can be advantageous to isolate the relevant component of the EGMs from the chamber of interest prior to calculating the inversely-solved map. As an example, separation and exclusion of the ventricular component (QRS) from atrial components can greatly benefit the quality of data produced by system 10 (e.g. mapping data produced by system 10). System 10 can perform various signal processing functions, such as to process ECG to inversely model QRST or PVCs (e.g. in a self-optimizing arrangement). In some embodiments, the body surface ECG can be used by system 10 to create a coarse model of ventricular depolarization and repolarization. In some embodiments, system 10 (e.g. via algorithm 115) can create this model using an inverse solution that dynamically detects ventricular activity (e.g. QRS) on the body surface leads, can calculate an inversely-solved estimation of ventricular activity (as would be measured at the location of the intracardiac catheter) for each ventricular beat, and can subtract the calculated ventricular activity from each corresponding beat of the intracardiac signals. The ventricular template can be identified automatically (e.g. by algorithm 115), and/or the template can be user-determined, based on intracardiac signals in addition to and/or in lieu of ECG-based parameterization. In cases where the ventricular component is inversely calculated by system 10 independently of the atrial component, the forward matrix can be estimated by system 10 (e.g. by algorithm 115) using a subject specific anatomy, a rule-based average, and/or a geometric primitive. This combination of estimation and subtraction is intended to preserve as much of the atrial component of the intracardiac signals as practical, while removing as much of the ventricular component as practical. The “atrial-only” EGMs can then be processed (e.g. by algorithm 115) using the inverse solution for whole-chamber non-contact mapping, yielding a map with minimal ventricular artifacts or mis-annotation due to the presence of ventricular artifacts. The ventricular estimation performed by system 10 can also be self-optimized by assessing the residual of removed ventricular signal and then updating the estimation accordingly to minimize the residual. Additionally, estimates may be refined by system 10 (e.g. via algorithm 115) by first applying grouping, clustering, and/or classification to ensure collected heart beats (also referred to as “cardiac beats” or “beats” herein) used for estimation are similar and well-matched to the beats to which the estimation is applied. Heart beats with different characteristics would be categorized as falling into a different group, and would thus have a different estimation to be applied.

System 10 can be configured to perform measurements (e.g. direct measurement) in an arrangement in which artifacts introduced by far-field activity are reduced. System 10 can make measurements from a first electrode (e.g. of a system 10 device inserted into a heart chamber), and use a second electrode (e.g. of the same or a different system 10 device) as a near reference. The first electrode can be configured to touch the tissue to make a measurement (e.g. configured to make a contact measurement). The second electrode can be configured NOT to touch the tissue when making a measurement (e.g. configured to make a non-contact measurement). By subtracting the second electrode's signal from the first electrode's signal, the far-field component measured by both will be suppressed, but the local signal measured only by the electrode in contact will be retained (e.g. and not be significantly suppressed).

System 10 can comprise one or more catheters or other devices that include stacked electrodes (e.g. stacked micromachined electrodes). For example, in some embodiments the first and second electrodes are configured in a stacked orientation, where the stacked electrodes are separated by a small separation distance. The electrodes in this configuration can be printed on a flexible circuit and can be deployed on a maneuverable catheter where in most deployment configurations, only the first electrode contacts tissue.

System 10 can be configured to perform measurements from far-field contributors, such as in the ventricle when treating and/or diagnosing an atria. In some embodiments, a ventricular estimation creation and subtraction process of system 10 can utilize one or more direct measurements from an opposing chamber. The direct measurements can be used in place of an inversely-solved template, or these measurements can be used to cross-check the inversely-solved template.

System 10 can comprise an algorithm 115 comprising one or more machine learning, neural net, and/or other artificial intelligence algorithms (“AI algorithm” herein), System 10 can comprise a machine learning algorithm or other AI algorithm (e.g. one or more algorithms of algorithm 115) for determining EGM fiducials, categories, and other parameters. System 10 can be configured to store, share, and/or learn from user-interventions (e.g. user-interventions of automated processes). System 10 can also be configured to adaptively self-optimize the automated processes in response to the learning. System 10 can maintain a self-updating dataset of any user inputs that override an automated detection or measurement made by the system. Choices by the user and the corresponding raw data can be treated as labeled data and stored. Some examples of such labels include but are not limited to: measurement channels to exclude; changes to thresholds to be made; changes to time-annotations to be made; beats to include and/or exclude; points to include and/or exclude; and the like. A stored database of the labeled data can be used to locally “learn” (e.g. identify or assess) the underlying features in the data that yield more accurate automation results. System 10 can also be configured to automatically share and/or transfer this database to a master repository that can be shared with one or more other system 10 units (e.g. at another location, such as when shared via a secure wired or wireless connection arrangement).

As described herein, system 10 can be configured to perform various forms of cardiac electrical mapping. System 10 can process cardiac signals to detect and analyze cardiac events, such as cardiac beats and/or cycles. System 10 can be configured to perform “rhythm tracking”, such as when cardiac beats are categorized. System 10 (e.g. via algorithm 115) can automatically differentiate and/or categorize each independent cardiac beat based on their having similar characteristics, either in real-time or near real-time (“real-time” herein), and/or in a post-processing step. The groups can be used for mapping modes that utilize multiple cardiac beats to aggregate data sequentially. Some mapping modes can utilize the beats that do not fit into pre-existing groups, and the function of beat grouping assists in automating the identification of these unique beats (e.g. via a “trigger mapping mode” of system 10 as described herein). System 10 can perform cardiac beat detection, annotating reference beats for an initial point in time, time T₀. In some mapping modes, system 10 time-aligns cardiac beats, such as on a time fiducial. System 10 can detect each beat by looking for a signal feature on one or more cardiac signals that are recorded by system 10. The cardiac signals analyzed by system 10 for beat detection can be unipolar, bipolar, omnipolar, Laplacian, and/or any mathematical combination of one or more signals. The analyzed signals can also be a derivative, an envelope, an energy function, a histogram, and/or other result of a mathematical operation performed on measured cardiac signals. System 10 can acquire these signals from one or more devices of system 10. A signal (e.g. a processed signal) can be the mathematical composite of multiple signals. System 10 can analyze more than one of the above signals (e.g. including processed signals) in combination, for example when system 10 analyzes both unipoles and bipoles (e.g. analyzes the unipoles and bipoles simultaneously or sequentially). Signal features identified by system 10 can include one or more features selected from the group consisting of: crossing a threshold (positive, negative, or absolute value); a local maximum or minimum peak (positive, negative, or absolute value); a local maximum slope (positive or upslope and/or negative or downslope). System 10 can use the time of the detected feature for T₀ alignment. System 10 can optionally apply a time offset from the detected feature. System 10 can use more than one of the above signal features in combination, for example a feature in which a cardiac event both crosses a threshold and has a sufficiently large local maximum negative slope. If system 10 is using more than one signal type (e.g. simultaneously), the signal features for each signal type can be the same or different for each signal type. In some embodiments, system 10 can require a peak positive of a filtered, bipolar signal to be of sufficient size in order to establish an independent beat and to establish a T₀ reference time. Alternatively or additionally, system 10 can use the mean, median, and/or max of an envelope or energy function of one or more bipolar signals to establish a T₀ reference time.

In some embodiments, conduction velocity is estimated as a global optimization function, whereby the global velocity is regularized and conditioned by system 10 (e.g. by algorithm 115) based on physiological ranges and loss functions that are defined based on deviations from average physiologic values and/or stratified based on a series of averages compiled from pathologic populations.

In some embodiments, the solutions are regularized by system 10 (e.g. by algorithm 115), such as by spatial filtering on graphs and/or meshes using techniques such as arithmetic mean, arithmetic median, and/or projection to graph-based basis functions. In the case of conduction velocity, the filtering can be applied to the coordinates of the velocity vectors in their canonical form and/or in their quaternion form. Filtering can also be applied via non-linear techniques, such as via neural networks and/or local non-linear filters, such as the median.

System 10 can be configured to perform a cardiac beat categorization in which reference channel exclusion is performed. System 10 can perform a reference channel exclusion selected from the group consisting of: excluding (e.g. automatically excluding) one or more channels; disabling (e.g. automatically disabling) one or more channels from use as a T₀ reference time; disabling (e.g. automatically disabling) one or more channels from use in detecting and/or categorizing cardiac beats; and combinations of these. These different channel exclusions can be made due to electrical disconnection and/or lack of detectable features on those channels, such as due to poor performance when used as a T₀ reference time and/or categorizing beats. System 10 can automatically disconnect one or more channels if a sufficient amount (e.g. a significant amount) of a specific frequency (e.g. 60 Hz) is present on that channel, as this condition can often be an indication that a sensor (e.g. an electrode) is electrically disconnected and/or “noisy” (e.g. experiencing electrical interference or other signal noise). System 10 can be configured to automatically exclude channels from use if the signal amplitude is below a threshold (e.g. if the sensor is in a suboptimal location to measure cardiac signals), and/or if the signal amplitude is above a threshold (e.g. when pacing results in a large pacing artifact that is much larger than the underlying cardiac signal). System 10 can be configured to exclude a sensor (e.g. an electrode) if its location is uncertain, inconsistent, and/or abnormal, as this can be indicative of a poor-quality electrical connection and/or an impact of an external system that could degrade performance. System 10 can be configured to automatically exclude channels based on: an analysis of statistics; data mining; and/or machine learning or predictive analytics to analyze the measured signals of that channel against a model and/or library of previously accepted and/or excluded signals (e.g. resulting in a determination of a set of channels to exclude). In some embodiments, the model and/or library used by a machine learning and/or predictive analysis algorithm of system 10 (e.g. an AI algorithm of algorithm 115) is comprised of labeled signals accepted and/or excluded based on operator decisions.

System 10 can be configured to perform “optimal reference channel selection”. System 10 can automatically select and/or suggest for use an optimal signal or set of signals to use for a T₀ reference and/or for cardiac beat categorization. System 10 can use ranges and/or other thresholds for amplitude and/or for timing interval (e.g. cycle length) stability and/or consistency. System 10 can utilize a confidence metric that is used to assess multiple signal characteristics including but not limited to: cycle length stability; amplitude; signal morphology (e.g. degree of fractionation or the presence of certain signal components, like a certain number of waveform deflections or a specific shape, like an “RS” morphology); and/or confidence score of a signal (e.g. when using only signals with a sufficient confidence score).

System 10 can be configured to compare various characteristics of recorded signals. System 10 can be configured to use one or more characteristics to differentiate or categorize cardiac beats, such as a differentiation based on a characteristic selected from the group consisting of: stability and/or consistency of timing interval (e.g. cycle length); signal morphology (e.g. unipolar signal morphology); envelope and/or energy function (e.g. example, bipolar signal envelope); timing sequence and/or pattern across multiple signals (e.g. the pattern of annotated time fiducials across a set of unipolar and/or bipolar signals from sensors at different locations in the heart); and combinations of these. System 10 can be configured to use a wavelet decomposition to isolate unique signal components from a signal's morphology, such as to quantitatively compare these components to those of other signals. System 10 can perform a cross-correlation between signals and/or wavelet components to quantify the degree of matching between two or more signals. System 10 can be configured to perform statistical analysis on the cycle lengths (interval between beats) to characterize beats. Similar cycle lengths can be indicative of the same rhythm or cardiac circuit, and differences in cycle lengths can be indicative of a change in rhythm or cardiac circuit. System 10 can be configured to use an envelope and/or an energy function of one or more signals. The signals can be unipolar, bipolar, omnipolar, Laplacian, and/or any other mathematical combination of one or more signals. When multiple signals are used by system 10, the envelope and/or energy function can be assessed for each individual signal, and/or it can be assessed for an aggregate or composite of the combined set of signals. For example, the envelope and/or energy function of each signal in a set of multiple bipolar signals from one or more locations in the heart can be used as a template against which the same signals from other cardiac beats can be compared. System 10 can be configured to “match” comparison signal sets that fit with the template (e.g. like a key into a lock). Alternatively or additionally, system 10 can stack, aggregate, and/or otherwise create a composite of multiple signals, and then determine an envelope and/or energy function of the composite to create a template. Then, for each comparison beat, a similar composite and envelope and/or energy function can be constructed and compared to the envelope/energy function of the template. In some embodiments, the cross correlation of the template envelope and/or energy function and the envelope and/or energy function from comparison beats can be used by system 10 to quantitatively compare beats. System 10 can be configured to match beats with a sufficient correlation score. In some embodiments, the combination of multiple characteristics can be used by system 10 to differentiate beats.

System 10 can be configured to separate cardiac beats into groups. For example, system 10 can be configured to automatically create unique groups and categorize individual beats into them. System 10 can perform multiple forms of clustering and/or classification approaches including, but not limited to: linear or quadratic discriminant analysis, correlation analysis, principal component analysis, K-means (e.g. connectivity or centroid based) clustering, support vector machines, kernel methods, neural networks, spectral clustering, hierarchical clustering, distribution-based clustering, density-based clustering, and/or grid-based clustering. These techniques can be configured to identify similar beats and/or to classify beats into groups. In some embodiments, system 10 processes a set of wavelet-decomposed signals using k-means clustering to determine a set of clustered groups of beats. System 10 can use combined and/or weighted scores of multiple signal characteristics to determine an overall group classification. For example, system 10 can group cardiac beats based on a combined weighted score using both cycle length and k-means clustering of wavelet-decomposed signals to produce (e.g. identify) multiple groups. By utilizing a weighted approach, system 10 can be configured to allow the user to select the relative weight assigned to each individual score. For example, a user may prefer to increase the relative weight provided for morphology scores, while down-weighting the importance of cycle length changes (or vice-versa) in the clustering/classification. System 10 can be configured to perform a live (e.g. real-time) and/or post-processed calculation. For example, system 10 can process the beat detection and classification as a post-processing step on recorded data, or iteratively, “on-the-fly” (e.g. live in real time) as each new beat is encountered.

System 10 can be configured in a “trigger mapping mode”, such as a mode configured to perform a “unique beat detection and rapid mapping routine” in which the routine detects unique cardiac beats and/or performs rapid mapping (e.g. based on PAC, PVC, and/or other triggers). System 10 can be configured to utilize (e.g. include in one or more analyses) the cardiac beats that do not fit into pre-existing groups, such as to identify unique beats and/or beats with few repetitions. System 10 can establish one or more beat groups, and then identify beats that do not match the groups that have been established. System 10 can be configured to visually designate (e.g. via graphical differentiation as described herein) and/or otherwise identify these non-matching beats (e.g. via a display of system 10). Once these unique beats are detected, several mapping methods can be employed by system 10 to identify signal-derived values (e.g. derived cardiac data) from these beats, including, but not limited to: activation times, peak-to-peak amplitude, beat or other inter-beat metrics wave timings (e.g. cycle length, S-T segments) and/or other data derived from the cardiac signals. Cardiac data can be calculated directly from the measured signals on one or more devices (e.g. catheters), and the data can be displayed as a visualization (e.g. a 3D visualization) on the anatomical shell. Deriving the cardiac data to generate the visualization from the cardiac signals (e.g. from the individual or plurality of catheters) can be performed by system 10 in a variety of ways including, but not limited to: interpolation and/or direct assignment of values based on proximity of the electrodes to the shell; solving of an inverse solution; and combinations of these. System 10 can be configured to differentiate (e.g. colorize or otherwise graphically differentiate) on the display: the sensors; objects (shell, markers, volume of space) in the vicinity of the sensors; and/or combinations of these. The differentiation displayed can be based on activation timing and/or amplitude data.

System 10 can be configured to perform a “rhythm classification routine”. For example, system 10 can utilize detected and categorized cardiac beats to make an automated “suggestion” of the rhythm type (e.g. as presented to an operator on a display of system 10). System 10 can base the suggestion on fixed metrics, such as cycle length ranges or R-R intervals between QRS (or any other inter-beat metric). System 10 can base the suggestion on: a statistical analysis; data mining; machine learning or other AI algorithm, and/or predictive analytics to analyze signals against a model and/or library of previously classified rhythms; and combinations of these. For example, cardiac signals, such as those used to detect and categorize individual beats (e.g. a set of reference EGMs from a catheter placed in the coronary sinus), can be processed by system 10 (e.g. via algorithm 115) using a wavelet transform that decomposes each waveform into different frequency bands while still preserving temporal information to produce a wavelet scalogram image. This processing of the cardiac signals can be performed by system 10 in an unsupervised workflow, as well. Image-based features in the scalograms of comparison beats can be assessed by system 10 using a convolutional neural network or other AI algorithm that is trained on a library of labeled scalogram images where the rhythm type is known. Some classifiable rhythms of system 10 include but are not limited to: atrial flutter, atrial tachycardia, atrial fibrillation (AF), sinus rhythm, paced rhythms, or ventricular tachycardia. Rhythm classification can also be performed using any duration of data (not just single, detected and categorized beats) and/or any set of signals acquired by system 10 (e.g. as acquired by reference catheters, mapping catheters, body surface electrodes, and the like).

System 10 can be configured to perform a mapping (e.g. cardiac mapping) of the present inventive concepts using various forms of data collection and data analysis. System 10 can be configured to display electrical events and/or activity (e.g. cardiac) in the form of electroanatomic maps (EAMs), based on data arising from electrical signals (e.g. electrograms or EGMs), where the data is visualized on a display of anatomical structure. The EAMs of system 10 can show activity data (AD) at a single location, a region, a chamber, the whole heart, and/or any other body volume (e.g. tissue volume). The AD can comprise activation times, amplitudes, conduction velocities, fractionation, complexity indices, pattern detections, sequence detections, causality indices, recurrence indices, dispersion indices, refractory (e.g. angle between subsequent activations) metrics, and/or any results of calculations from signals (e.g. cardiac signals and/or imaging signals). Similarly, integrated metrics, such as metrics using the activation sequence in conjunction with refraction metrics can be applied by system 10 (e.g. by algorithm 115) to determine a spatiotemporal initiator of the refractory event. The EAMs produced by system 10 can also include AD based on signals from the interior surface (endocardial surface), from the exterior surface (epicardial surface), and/or from tissue between the two surfaces (mid-myocardial or trans-myocardial tissue). The AD can be based on signals acquired by electrodes in contact with the tissue (contact data) and/or the AD data can be from calculated signals at the tissue derived from electrode measurements not in contact with the tissue (non-contact data). The AD can be based on signals that can be unipolar, bipolar, omnipolar, Laplacian, and/or any other mathematical combination of one or more signals, and/or from a derivative, an envelope, an energy function or other result of a mathematical signal operation. The AD can be based on signals that can be acquired from one, two, or more devices of system 10 (e.g. one, two, or more catheters, patches, and/or other components of system 10). Both contact mapping and inverse-solution-based non-contact mapping are described herein. The AD can be based on contact-measured and/or non-contact calculated signals. The EAMs can comprise a combination, aggregation, integration, and/or fusion of one or more types of map data, for example AD of unipoles and bipoles simultaneously, and/or AD of contact and non-contact signals simultaneously. System 10 can also include a “data fusion algorithm” (e.g. one or more algorithms of algorithm 115) that can be configured to cohesively combine data of different forms and/or of different signal origin. For example, the data fusion algorithm can calculate activation times from: both bipolar signals and from corresponding unipolar signals; and/or from contact signals and non-contact signals. The data fusion algorithm can determine if either, both, or none, are viable, and if both are viable, determine the activation time to use in the EAM. If the activation times disagree, the data fusion algorithm can use rulesets involving different signal characteristics, such as amplitude, slope, width, morphology, energy, and the like, to select the optimal activation time to use, and/or to calculate an intermediate value to use. In these embodiments, system 10 comprises one or more thresholds that are used to assess signal viability or to perform another data assessment. Alternatively or additionally, the data fusion algorithm can include a learning model that is configured to determine the optimal activation time to use based on historical data (e.g. labeled data), and/or through the use of an unsupervised workflow (e.g. a workflow that uses unlabeled, non-fiducialized data). In some embodiments, the data fusion algorithm can combine contact bipolar amplitudes with non-contact unipolar amplitudes. In other embodiments, the data fusion algorithm can combine activation times from contact signals (e.g. contact bipolar signals) and from non-contact signals (e.g. non-contact unipolar signals). The data fusion algorithm can establish the relationship between data of two different types and/or different signal origin and provide the data for display in a cohesive unit of measure, for example a normalized percentage or an equivalent unit of measure.

System 10 (e.g. via the data fusion algorithm) can be configured to perform mapping directly from measurements, such as to perform “live scanning” (e.g. scanning in real-time). System 10 can be configured to directly calculate electrical activity information from measured signals, and to display them (e.g. rapidly display them) on the anatomic structure (e.g. a shell or other image of the anatomic structure provided on a display by system 10, as described herein). In some embodiments, the data is assigned or projected to the anatomic surface on a display of system 10. Electrical activity information can be shown on the anatomic structure when collected in close proximity (e.g. a distance less than 5 mm) to the anatomic structure. System 10 can calculate (e.g. directly calculate) electrical activity information from electrograms at a distance (e.g. a threshold distance of at least 5 mm) and project to the anatomic structure as a sparsely-sampled, low-density map of the electrical activity information. In some embodiments, system 10 comprises an “interpolation algorithm” (e.g. one or more algorithms of algorithm 115) that is configured to calculate the displayed data in areas where measurements are not available. The data fusion algorithm can cohesively integrate AD collected in close proximity with AD measured at a distance (e.g. a distance above a threshold). In some embodiments, unipolar signals from a device (e.g. a catheter) that is not in contact with the tissue (e.g. the electrodes are not in contact with tissue) are measured and directly annotated for local activation time, for instance, using the steepest negative slope. These activation times are projected to the nearest surface or the nearest surface along a vector (such as a vector normal to the orientation of an electrode). When the device (e.g. its set of associated electrodes) is positioned near-central in the chamber, the activation times can project around the entire chamber. When the device (e.g. its set of associated electrodes) is closer to a wall of the chamber, the activation times can project to the close wall. The activation times can be calculated by system 10 for every detected cardiac beat, and/or these times can be calculated for detected unique beats only (e.g. as described in reference to the trigger mapping mode and/or the unique beat detection and rapid mapping routine herein). Once AD has been calculated by system 10, as another visualization, images of the measuring sensors (e.g. electrodes) can be differentiated (e.g. colorized or otherwise differentiated on a display) to show the relative relationship between each sensor. For instance, if the AD is the local activation time, the earliest-detecting sensor can be color-coded to red, designating an “early” part of the circuit, while the latest-detecting electrodes can be color-coded to purple, designating a “late” part of the signal. Alternatively or additionally, the AD can be shown on the anatomic structure and can be color-coded similarly. Other variations of visual differentiation are within the spirit and scope of this application.

System 10 can be configured to automatically place a “marker of interest” on a display of information provided by system 10 (e.g. mapping and/or other information relevant to a clinical procedure performed using system 10). System 10 can be configured to place a marker of interest (also referred to as “marker” herein) in the displayed coordinate system, and/or on the anatomic structure, such as a placement at notable locations based on the AD. The marker can have visual attributes that designate the degree of confidence of the marker (e.g. confidence related to the noted location). For example, if the AD is the local activation time, system 10 can display a large marker on the anatomic shell at the “earliest” location. For each detected cardiac beat, a new large marker can be placed on the anatomic shell. The operator may interact with the marker (e.g. via a user interface 120 of system 10), such as to show (e.g. add) relevant information about the corresponding beat. Over many successive detected beats, system 10 can provide (e.g. visually provide) a number of markers, such as when these markers indicate the spatial consistency of marked locations.

System 10 can be configured to adjust the resolution of a map (e.g. an EAM), such as to change to (e.g. upgrade to) a high-resolution map (e.g. from a lower resolution map). When used with the unique beat detection and rapid mapping routine described herein, the maps and markers of each unique beat can be used to show the earliest site of activation. The map data may be somewhat coarse when directly calculated. However, any directly calculated map of a detected beat can be further processed to a high-resolution map using the inverse solution. The corresponding displayed color map and the large marker will become more detailed, the EAM will be calculated with higher resolution, and the marker will become smaller and more precise in location. The direct calculation of AD to form a map, automatic placement of a marker, and further processing to a high-resolution inversely-solved map can be performed by system 10 on any beat designated by an operator.

System 10 can be configured to provide a confidence score related to a chamber of origin. System 10 can be configured to perform a “chamber of origin routine” (e.g. via algorithm 115) that provides a confidence score that the origination point of a given beat (e.g. if one exists) lies in the chamber being mapped, or in the adjacent chamber. The Chamber of Origin Routine can use relative timing information between intracardiac measurements and body surface measurements to calculate the confidence score. The routine (e.g. algorithm 115) can also use one or more morphology analyses to automatically identify characteristic morphology features in the EGMs at the earliest sites of activation, for example, a slight positive hump of an rS pattern, such as to be used by system 10 to determine the confidence score.

System 10 can include an algorithm 115 that is configured to perform a “regional inverse mapping routine”, such as a routine in which an inverse calculation of cardiac activity within a region is performed. The EAMs calculated by system 10 can be whole chamber inversely-solved maps. In some embodiments, the inverse solution is applied to solve for EGMs that apply to only a portion of the chamber. The calculated EGMs in the region can be used by system 10 to determine AD in the region, including activation times, signal amplitudes, and/or scar regions. The forward matrix can be adapted (e.g. by algorithm 115) to solve for the entire chamber, such as when the entire chamber is missing certain structures (e.g. missing the left atria appendage), and/or to solve for those individual structures themselves. Using the forward matrix, the inverse solutions can be derived (e.g. by algorithm 115): via a direct inverse method, by solving a system of equations, via one or more neural-networks, and/or via iterative solutions of a regularized optimization problem. These optimizations can include the residual term, which can enforce consistency with the measurements and regularization terms (e.g. and impose regularity or prior knowledge about the inverse solution). The residual terms can use a variety of loss metrics including least-squares, sum of absolute differences, and/or re-weighted least-squares. The residual term can also be used by system 10 to constrain the solution to fulfill specific constraints, such as being temporally non-decreasing or non-increasing, or residing within a predetermined subspace (e.g. a subspace derived from the data via linear or non-linear methods and/or derived from the domain where the solution is defined, such as graph-based basis functions). The regularization terms used by system 10 can be in the form of zero, first, and/or second order Tikhonov regularization. Alternatively or additionally, other regularization methods can be used, such as graph-derived basis functions, dictionary-based approaches, median filtering, plug-and-play methods, and/or neural-network derived regularization.

In some embodiments, system 10 can directly estimate the relevant AD information without explicitly deriving the EAMs first. This estimation can be performed by system 10 either through projection and interpolation of the AD from the catheter-captured signals (e.g. signals captured by one or more catheters as described herein) onto the chamber (and/or a region thereof) and/or through non-linear iterative optimization, such as to directly solve for AD given a predetermined model for the electrical activity on the regions of interest.

Post processing approaches can be applied by system 10 (e.g. by algorithm 115) to both EAM and AD solutions with a variety of methods, including mean and median filtering, graph-based filtering methods, and/or neural-network approaches.

System 10 can include an algorithm 115 that is configured to perform a “supermap routine”, such as a routine configured to produce an activity map derived from signals made over time and recorded from an electrode array that is repositioned during the recording process.

System 10 can be configured to perform a cardiac information analysis. System 10 can process activity data (AD) to calculate additional metrics that can be used to analyze the electrical activity of the patient's tissue. Various analyses can be performed by system 10 (e.g. by algorithm 115) to identify one, two, or more clinical sites of interest. One analysis that can be performed by system 10 is the identification of conduction patterns by system 10 (e.g. by algorithm 115) using one or more of a variety of path finding techniques, such as streamlines and/or other techniques that identify a clinically relevant pathway, this identification process herein referred to as “autopathing”. System 10 can perform autopathing by taking a series of streamlines and clustering their traversal using AD (e.g. electrophysiologic data and/or biophysical data) to determine a descriptive pathway of electrical propagation across a given heart chamber (e.g. atrial body). This process is an extension to a streamline-based technique that determines nearly all pathways (e.g. given enough starting locations). Some analyses performed by system 10 (e.g. by algorithm 115) can be configured to quantify the spatial distribution and/or temporal occurrence rate of one or more features of interest, such as to characterize one or more clinical sites of interest. The autopathing performed by system 10 can operate within an altered coordinate system (e.g. in addition to the cartesian coordinates). System 10 can represent the data in a 2D conformal data space and/or in an anatomically determined data space that is created from the subject-specific anatomy (e.g. universal atrial coordinates). Some analyses performed by system 10 can quantify the spatial distribution and/or temporal occurrence rate of features of interest, such as to characterize the clinical sites of interest. Feature identification includes, but is not limited to: block; isolation; isthmus; breakthrough; and/or epicardial bridging. The feature “block” can represent the annihilation of activity at a location, with no coherence to collocated sites. The feature “isolation” can represent that a location of contiguous tissue has been electrically isolated from another region of tissue (such as the pulmonary veins being electrically isolated from the body of the left atria). The feature “isthmus” can represent a region of tissue in which collocated, but not necessarily contiguous, pathological tissue is present, and in which reentrant phenomena are made more likely. The feature “breakthrough” can represent a surface location (e.g. a surface such as the endocardium) in which the origin of electrical activity has not originated on that surface location (e.g. the earliest site of activity on that surface but not within that structure). The feature “epicardial bridging” can represent a conductive path of tissue proximal to tissue that may be stunned or ablated.

System 10 can be configured to identify various conduction patterns, such as localized irregular activation (LIA), localized regional activation (LRA), and/or focal patterns. System 10 can be configured to detect and count the occurrences of spatiotemporal conduction patterns anywhere in a chamber (e.g. a heart chamber such as the left atrium) by analyzing the spatiotemporal activation sequences present in activation maps. At every location in the chamber (vertex on the mesh), each activation at that location can be analyzed by system 10 in context with neighboring activations within a small surrounding region (a diameter of at least 5 mm or 10 mm and/or a diameter of no more than 25 mm or 15 mm). Conduction velocities can be calculated from activation times within this region. The activation sequence and conduction directions of every beat can be assessed against one or more sets of rules to categorize the local conduction pattern. The occurrence rates of each pattern type at each location can be quantified and can be displayed as a histogram (e.g. a color-differentiated histogram) on the anatomic model (e.g. the shell), where higher rates of occurrence in the same location can be visualized with differentiated visual properties (e.g. greater opacity and color intensity). Visualizations of occurrence rates for multiple pattern types can be displayed by system 10 simultaneously.

System 10 can be configured to determine various conduction properties, such as when activation data (AD) is processed to quantify different conduction properties. System 10 can be configured to determine conduction velocity. The conduction velocity through tissue is a highly relevant metric of tissue activity. Spatially and/or temporally-distributed local activation times can be used by system 10 to calculate local conduction velocity. In some embodiments, local activation times on a 3D shell surface mesh can be projected to a plane, and the spatial gradient of activations in the projected plane can be used to approximate conduction velocity. Similarly, conduction velocity can be calculated by system 10 (e.g. by algorithm 115) in an elementwise arrangement using similar operations (e.g. gradient estimations on computational elements), such as to selectively augment performance in some areas (e.g. increase sensitivity to small spatial structures). The gradient operation can be performed by system 10 using a triangulation and/or finite-differences type approach for estimating the gradient. Conduction velocities can be color-mapped (e.g. where data is presented in a color-coded or other graphical differentiation arrangement) and/or the velocities can be displayed directly. Deceleration can be calculated and displayed as a conduction metric by calculating the gradient of conduction velocity. Relative conduction velocities can be determined by system 10, such as a conduction velocity determined (e.g. and provided to an operator) as a percentage change (e.g. decrease and/or increase) and/or as a normalized value. Relative conduction velocities can be an advantageous metric to account for potential patient-to-patient or map-to-map variations in conduction velocity. Often, identifying the regions of greatest acceleration (e.g. greatest slowing or increasing in velocity) can be more clinically relevant and valuable to diagnosing an arrhythmia (e.g. AF) than crossing a specific threshold of velocity. Relative conduction velocities can be calculated as a percent decrease or increase, and/or the velocities can be normalized to the fastest percentage of velocities in the map. Refraction of the wavefront can also be estimated by system 10 (e.g. by algorithm 115) using the conduction velocity estimated throughout the chamber. Refraction can be based on the angle between subsequent activations of a region of tissue. Using any of these parameters, in conjunction with the activation sequence, the site of a refractive map can thereby be estimated by system 10 (e.g. by algorithm 115). System 10 can be configured to determine a signal amplitude. For example, system 10 can use the amplitude of local signals as a metric of conduction. In some embodiments, the peak-to-peak amplitude of local bipolar signals can be color-mapped (e.g. a map that differentiates by varying color and/or other graphical property). In some embodiments, the peak negative amplitude of local unipolar signals can be color-mapped. In some embodiments, an omnipolar or Laplacian amplitude can be color-mapped. In some embodiments, the amplitude of non-contact-calculated charge density signals can be correlated with amplitudes of contact voltage signals in the same location. The correlation can be used to define a representative relationship between charge density units and voltage units for that map. In some embodiments, this relationship can be used to create amplitude maps of both charge density and voltage data types. The representative relationship between the charge density calculation and the millivolt equivalence can be calibrated by system 10 (e.g. by algorithm 115) using inversely computed potentials (e.g. potentials that are directly sampled signals near the anatomical body in the blood pool).

System 10 can be configured to perform a spatiotemporal analysis. Numerous forms of spatiotemporal analysis can be performed by system 10. Map data of system 10 can comprise a set of spatially connected time-varying signals (electrograms) and/or temporal events (e.g. local activation). System 10 can determine a multi-dimensional activation sequence or pattern. In some embodiments, the spatial distribution of time events can be plotted (e.g. and displayed) by system 10 as a multidimensional image, where time is a first dimension, and the spatial distribution of anatomical locations can be 3 additional dimensions, or can be reduced to a smaller number of dimensions (e.g. through projection to a 2D parameterized space, or dimension reduction by mapping to a universal common coordinate, universal atrial coordinates for atrial EAMs). The reduced data space can also be computed in a conformal data space in which the mitral valve is used by system 10 (e.g. by algorithm 115) as the point of unwrapping. The time-varying nature of electrical activity can therefore be captured as a spatiotemporally-representative static image (SRSI), where the size of the image is largely governed by the time duration of the map data. This technique therefore allows the highly complex multi-dimensional data set to be processed by system 10 using image analysis and/or comparison techniques. In some embodiments, the SRSI can be analyzed by system 10 by searching for kernel patterns of a given window size in the time-dimension that repeat in other parts of the image. These repeating recurrences can be clinically relevant in characterizing the activity and targeting therapy. Window sizes can be varied from very small to very large to reprocess the SRSI multiple times to search for possible kernels of different size. In some embodiments, the SRSI can be analyzed by system 10 for spatiotemporal coupling relationships between different areas of the anatomy. In the SRSI, the activation sequences between two regions of the chamber that have a strong coupling relationship will follow common and consistent vectors that can be detected by numerous pattern detection techniques, including machine learning, deep learning, and/or other AI algorithm. In some embodiments, the coupling is determined by system 10 (e.g. by algorithm 115) via an analysis of the temporal variation in the spatial correlation.

System 10 can be configured to perform a spatiotemporal analysis by performing a network analysis. In some embodiments, the spatiotemporal sequence of activations can be analyzed by system 10 as a network analysis. The network can be formed by interconnected nodes of the anatomy, where neighboring nodes are adjacent locations on the anatomy, and nodes further apart are further apart along the anatomy's surface. For any activation at a node, the upstream and downstream activations encode coupling relationships between areas of the anatomy, and “bottlenecks” through which downstream activation pervades large areas of the anatomy can be efficient therapy targets to modify or eliminate a perpetuating rhythm. In some embodiments, each activation at each node can be assessed within a window (e.g. a window of at least 25 ms or 50 ms, and/or a window of no more than 250 ms or 100 ms), such as to assess the area of downstream influence of activation at each node. Areas of greater downstream influence can be more effectual in the perpetuation of an arrhythmia.

System 10 can be configured to analyze a spatiotemporal sequence of activations by visualizing the time-referenced activation area. The activation time data in an EAM can be broken into time intervals by system 10 (e.g. by algorithm 115). The time-referenced activation area can be calculated as the area corresponding to the portions of the EAM with activity times within each time interval. Alternatively or additionally, the number of measurements and/or points can be used by system 10. The time-referenced activation area can be provided (e.g. visualized) as a plot, for example as a histogram with one axis representing time and the other axis representing the area of activation. In some embodiments, more than one visualization can be displayed at once by system 10, using the same time-axis. Each visualization can show data from different EAMs. Each visualization can alternatively show data from the same EAM, but indicate the form of the different data types, for example when displaying (e.g. and graphically differentiating) non-contact data, contact data, unipolar data, and/or bipolar data.

Similarly, system 10 can be configured to analyze a spatiotemporal sequence of activations via amplitude-referenced activation area visualization. The activation time data in an EAM can be broken into amplitude ranges. The amplitude-referenced activation area can be calculated as the area corresponding to the portions of the EAM with activity times within each time amplitude range. Alternatively or additionally, the number of measurements and/or points can be used. Visualizations similar to those as described hereabove can be used by system 10 to display the amplitude-referenced activation area.

System 10 can be configured to perform a cardiac information analysis comprising a data aggregation and statistical analysis. Single data sets can be vulnerable to false positives and negatives, particularly when selected metrics can incur bias solely from the measurement of the metric itself. In some embodiments, system 10 comprises a bias (e.g. a user configurable bias) that causes analysis to tend toward and/or away from false positives and/or false negatives. The amplitude of bipolar (a subtraction of one unipolar signal from another) signals is commonly used as a surrogate for measuring tissue abnormality, and it is typically measured just once. However, measurement orientation, wavefront direction, and tissue rate response all affect the amplitude of the bipolar signal, rendering it a non-specific metric for tissue abnormality. In some embodiments, system 10 is configured to overcome one or more of these limitations by performing multiple measurements, varying the wavefront direction and tissue rate response to remove the potential intrinsic biases. Once these multiple measurements are made (e.g. using system 10), understanding the spatial consistency of any metric of abnormality improves the specificity of detecting the abnormality. System 10 can be configured to make a composite from multiple measurements. For example, multiple measurements (activations in one or more maps) can be made by system 10 under varying conditions. Each measurement can comprise activity data (AD) at a common set of locations on the anatomy (vertices of the mesh). With multiple measurements (activations in one or more maps), each location on the anatomy possesses a composite set of data samples that can be statistically analyzed by system 10. The composite dataset is an aggregation of multiple datasets into a commonly analyzable structure. In some embodiments, the structure is the mesh of vertices of the anatomical structure. In some embodiments, the AD assessed as a composite is conduction velocity. In some embodiments the AD assessed as a composite is signal amplitude. The data in the composite map can be statistically analyzed or assessed using thresholds. For example, a composite data set can be used to visualize the minimum, mean, max, and/or median conduction velocity and/or amplitude at all locations in a heart chamber. System 10 can be configured to perform a consistency analysis. For example, the composite data can be consolidated by system 10 by applying thresholds. For instance, if a conduction velocity threshold (e.g. a threshold of 0.3 m/s) is used as a threshold for abnormal conduction (slower is typically more abnormal), then the composite data could be assessed by counting any conduction velocity (CV) in the composite data set less than the threshold as abnormal, and/or any CV greater than the threshold as normal. A consistency map can then be displayed showing areas with consistently abnormal CV, consistently normal CV, or inconsistently abnormal CV in a visually differentiating fashion (e.g. via color-coded visualization). In some embodiments, CV is thresholded by system 10 to form a consistency map. In some embodiments, signal amplitudes are thresholded by system 10 to form a consistency map. In some embodiments, the threshold for abnormal activity is combined between CV and signal amplitude by system 10 to form a consistency map. In some embodiments, multiple metrics or thresholded metrics can be combined into a score for each activation by system 10, which can then be displayed in the composite map.

As described hereabove, system 10 can be configured to perform one or more cardiac activation analyses, such as when performing a “fusion of clinical measurements and/or computational modeling”. System 10 can be configured to perform “anatomic data co-registration” such as via a “universal anatomic model” and/or “landmark co-registration” (skeleton). Anatomic data co-registration performed by system 10 can comprise system 10 (e.g. algorithm 115) assuming a fairly consistent collocation of the four chambers of the heart, and in scenarios where subject-specific (i.e. patient-specific) orientation cannot be determined, the relative positioning of various chambers can be determined via population level averages (e.g. averages from a sample of human subjects). A universal anatomic model of system 10 can utilize a global cardiac positioning system to collocate chambers of the heart relative to one another. Landmark co-registration of system 10 can utilize a calculated skeleton that traces the relationship between the four chambers of the heart in 3D space, and/or a series of 2D cross sections, such as to determine the relative orientation and positioning between the relative chambers. System 10 can be configured to perform “CV aberration/divergence modeling”, such as when system 10 makes a measurement (e.g. a clinical paced measurement) from one or more locations (e.g. using the supermap routine described herein and/or a single position routine). The activation can be mapped and analyzed to find areas of block (e.g. isolation). System 10 can use the same chamber anatomy (e.g. as previously calculated and/or presented), and computationally apply the areas of block. A “restitution score” determined by system 10 can comprise a score determined by analyzing the conduction velocity at a range of sites across varying pacing rates such that subject-specific restitution information can be developed to thereby parameterize subject-specific simulations or compare the degree of restitution related change to population averages as an index of restitution (e.g. restitution score). System 10 can use a model of simulated propagation to calculate the chamber-wide activation sequence. The model can be isotropic. In some embodiments, the model is parameterized by system 10 (e.g. by algorithm 115) based on the measured activation and thereby system 10 can initiate the simulation from varying points in the measured activation. For example, system 10 can use the measured first 10% of activation, which can be compared to the remaining 90% of measured activation against the simulation. This process can elicit a number of properties of propagation that show anisotropic-like divergences which could be due to fiber orientation or substrate related changes. Alternatively or additionally, the model can be anisotropic (e.g. with heterogeneous properties) and/or the model can be determined based on a population average and/or a population atlas. Heterogeneities of the model can be based on: a standard model; and/or data from one or more measurements, for example data acquired from a CT and/or an MRI. For example, a fibrosis score and/or arrhythmogenic score based on the spatial configuration and/or substrate specific changes to the intensity in the CT and/or MRI data, can be used by system 10. These intensity changes can be elicited via contrast agents or through standard imaging regimes and/or an analysis of clinical maps, for example composite maps of conduction velocity. A series of conduction velocity maps can be used by system 10 to estimate the fiber orientation across the heart chamber (e.g. an atrial body), and these estimations can then be used to produce anisotropic simulations that take into account the fiber orientation. In some embodiments, differences between the anisotropic simulations and the measurements tend to indicate substrate-related differences not taken into account by the simulation framework of system 10. System 10 can compare clinical paced maps and simulated propagation to determine differences in the conduction behavior. System 10 can calculate directional divergence between clinical and simulated maps, such as to show preferential conduction directions (e.g. those that might exist from anisotropic fiber orientations) or substrate related changes. As described hereinabove, the estimation provided by the system 10 simulation framework (which is in itself parameterized by the EAMs or population averages) can be compared to the measurements themselves, such as to determine areas of maximal divergence which may indicate abnormal tissue. System 10 can calculate aberrations in the clinical map (e.g. aberrations that are different from the simulation framework in terms of speed, restitution, initiation, breakthrough, and/or other propagation related phenomena). System 10 can perform simulated pace mapping to find gaps, such as to: track delivered therapy and all therapy parameters; model the effects of delivered therapy on the local conduction (e.g. local conduction can be unaltered, partially/moderately altered, or completely eliminated (no conduction)); and/or simulate initiation of activation from one or more areas that engages with the delivered therapy. For example, based on one or more system 10 thresholds (e.g. user-defined thresholds), a series of standard settings, or a combination of the two, system 10 can produce an electrical propagation simulation (e.g. subject specific, rule based, or a combination) to track delivered therapy. Additionally, system 10 can be configured to model the effects of delivered therapy on the local conduction. Local conduction can be unaltered, partially and/or otherwise moderately altered, or completely eliminated (e.g. no conduction), and as such, can be parameterized by system 10 (e.g. by algorithm 115) in physiological simulations as described herein. Parameterization performed by system 10 can take into account default geometric parameters (e.g. tissue thickness), electrophysiological parameters (e.g. restitution curve data), and/or anatomical measurement data.

System 10 can comprise a “data management architecture”. The system 10 data management architecture (DMA herein) can comprise an arrangement in which case information (e.g. information captured and/or related to the clinical procedure being performed on a patient) is spatiotemporally stored. Broadly, DMA can include a volumetric regular grid large enough to encompass a typical four chamber heart. This architecture can be formed of a regular structured rectilinear grid, as an unstructured tetrahedral grid, and/or as an unstructured hexahedral grid. The data storage can include: one or more co-referenced data spaces; and/or a hierarchy of data spaces (e.g. parent, peer, child, and the like). System 10 can perform data processing in each data space. System 10 can recalculate (e.g. refactor) data in “child” data spaces, such as a recalculation based on a “parent” data space. System 10 can provide visualization of these various data (e.g. measured, determined, and/or calculated data).

Therapeutic and navigational information can be stored by system 10 in an element-by-element basis, such as for further processing and/or analysis performed by system 10 and/or an operator using system 10. One or more co-referenced data spaces, such as domains within the DMA, can be computationally-associated with one another, such as to facilitate two chamber mapping. In some embodiments, it may be appropriate that the geometry is displaced more than may be physiologically appropriate, but system 10 can be configured to constrain the biophysical activity to reflect the true physiological configuration. These data spaces can be: provided (e.g. displayed), obscured, highlighted, and/or augmented (e.g. on demand by a user of system 10) by system 10.

Hierarchy of data spaces (e.g. in a parent, peer, child arrangement) within the data space can exist, whereby several independent geometries can comprise dependent properties that can be inherited based on examination in one geometry (e.g. in one portion of a geometry), but not another. These computational property-inheritance mechanisms can be dictated and facilitated by system 10 by the various geometries' relationship to one another. For example, the left and right atria can have a peer relationship in which there is relatively minimal inheritance of properties. Alternatively, the left atrial appendage can be classified by system 10 as a child of the left atrium, and the parent-child relationship can have a much more substantial inheritance. Data processing in each data space can utilize the DMA to regularize and constrain data manipulation, such as to facilitate whole-chamber to whole-organ level analytics and visualizations. For example, the DMA can be used to: constrain global electrical solutions across the bi-atrial geometry; constrain geometric manipulations and perform “shaving” of the heart chamber (e.g. one or both atrial bodies); and/or solve a piecewise inverse problem that optimizes for the bi-atrium.

Refactoring data in “child” data spaces based on “parent” data space can comprise changes based on specific criteria, and certain parameters can be delegated by system 10 (e.g. by algorithm 115) to the peers and/or children of certain geometries, the delegation based on measurements and/or parameters changes in that geometry and/or anatomy.

Visualization of data by system 10 can comprise a volumetric grid that is used to facilitate 3D visualizations that may be only partly associated with the underlying cardiac mesh. These visual elements can be scalar, vector, matrix, and/or tensor-based visualizations and/or analytics. Additionally, the visual elements provided by system 10 can utilize variations in color, size, shape, and/or other variable graphical parameter to connote properties of the tissue, the organ, and/or the accuracy and/or confidence in a given metric.

Establishing a finite element method (FEM) framework for subject-specific or atlas-based simulations can comprise the use of the structured and unstructured mesh comprising the DMA and a series of FEM based analyses. Within the DMA, system 10 can perform bidomain, monodomain, pseudo-bidomain, eikonal, reaction-eikonal, and courtemanche-type simulations, such as when the simulations are performed by system 10 on one or more of the anatomical chamber models produced by system 10. These computational models can be parameterized by the subject specific AD, and/or the models can be strictly rule-based and/or population based.

System 10 can be configured to perform event-driven user interface control and data representation. System 10 can provide “data elements” of information, throughout a clinical procedure, and system 10 can track these data elements, such as when tracking one, two, or more data elements selected from the group consisting of: recorded signals; user actions; clinical events; EAMs; delivered therapy; rhythm classifications; beat groups; beat characteristics; and combinations of these. Each data element can be saved with time information (e.g. a time stamp). Data elements can be displayed in a chronological timeline. Data elements of different data types (e.g. as categorized by algorithm 115 of system 10) can be displayed in the timeline synchronously. For example, data collection, beat groups, created EAMs, delivered therapy, markers, collected anatomy, case events, and/or time calipers can be shown in different parallel tracks in a synchronous timeline. Each track can be graphically differentiated (e.g. color-coded or otherwise graphically differentiated) corresponding to user interface representations used in and/or for the rest of system 10. System 10 operations can be initiated via one or more particular user actions (e.g. clicking on an icon or pressing a hotkey), which can at a minimum log the time and/or mark a fiducial visualization in the timeline as an “event”. Additional information can be added to the event, such as a text label and/or notes. The event can be modified to other forms of data, included time calipers, an EAM, a beat label, a beat group, an anatomic marker, and/or a label. In review, a user (e.g. a clinician or other operator) can return to the event on the timeline and observe the system environment (e.g. system parameter levels, patient physiologic data, and/or other information) that existed and/or otherwise was relevant at that moment in the clinical procedure. Operations of system 10 initiated through a user interface of system 10 can also be logged as an event on the timeline, with the corresponding attributes of the operations applied to the event automatically. User actions and changes can also be stored as events, including actions and changes such as to application settings (e.g. modifications), calculation parameters, applied filters, creation of data entities (like anatomy components, beat groups, text labels, and/or graphical markers), changes to anatomy data (e.g. shaving, cutting, and/or adding anatomy), and the like. Event information can also be displayed as a corresponding log or list. In some embodiments, a chronological representation of event data can be used to undo or redo user actions through a user interface, such as a graphical user interface (GUI), GUI 125 of system 10 as described herein, such as by dragging an indicator to exclude previous user actions or clicking on previous actions and deleting them. Undoing previous events can be performed on a contiguous set of events leading up to the current state of the system. Events can be deleted asynchronously, and if deletion of an event asynchronously requires associated events to also be deleted, system 10 can notify the user and visually designate the associated events prior to deletion.

FIGS. 2 through 8B herebelow illustrate various examples of the present inventive concepts described hereabove.

Referring now to FIG. 2, an embodiment of an anatomic model representing a tissue surface is illustrated, consistent with the present inventive concepts. System 10 can be configured to display an anatomic model representing the walls of a cardiac chamber and/or another tissue surface of a patient. The anatomic model can be displayed to the user (e.g. a clinician of the patient) via a user interface, such as via GUI 125 shown and described herein. Portions of the anatomic model can comprise varying graphical properties, such as varying color intensity, opacity, or another variable graphical parameter used to differentiate data. The varying properties can represent variations in the anatomic model, for example variations in the density of data collected to calculate a portion of the anatomic model. In some embodiments, system 10 can display one or more points proximate the anatomic model, such as one or more points representing data collected by system 10. For example, system 10 can display points representing ultrasound data points collected using one or more devices configured to transmit and/or receive ultrasound signals (such as is described herein).

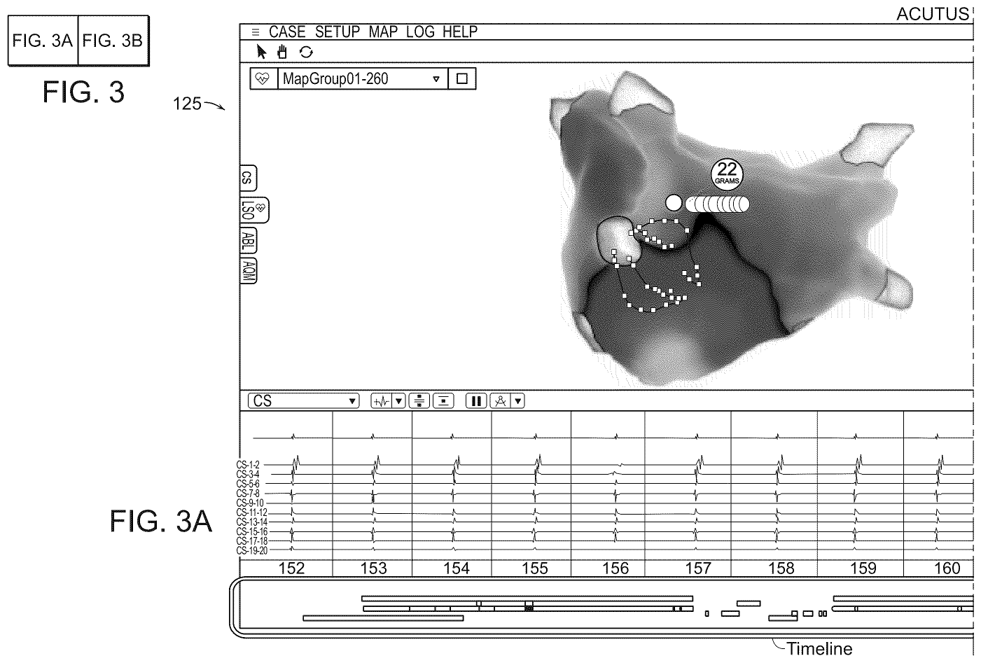

Referring now to FIG. 3, an example of a graphical user interface displaying cardiac mapping data is illustrated, consistent with the present inventive concepts. System 10 can be configured to provide a graphical user interface, such as GUI 125 shown. GUI 125 can comprise multiple display areas configured to present information collected and/or calculated by system 10 to the user. Information can be displayed to the user via one or more graphical representations, such as representations selected from the group consisting of: visual models, such as 2D and/or 3D models; graphs; charts; timelines; overlays; icons; other visual representations of data (e.g. system 10 device geometry, orientation, and/or other position data); and combinations of these. GUI 125 can display information related to one or more cardiac beat groups identified by system 10 as described herein. In some embodiments, the beat groups can be graphically differentiated (e.g. color-coded), based on one or more unique characteristics of the beat group, for example the morphology, cycle length, time sequence, energy profile, and/or other one or more characteristics. In some embodiments, each beat group identified by system 10 is indexed (e.g. assigned an integer value). In some embodiments, one or more recorded beats that have not been assigned to a beat group can be displayed with a unique graphical property (e.g. a unique color and/or pattern).

In some embodiments, GUI 125 includes a “timeline view”, as shown in FIG. 3. A timeline view can display various data types relative to a timeline (e.g. in parallel relative to a timeline). For example, the timeline view can indicate when data was collected and/or for what time periods results have been calculated. The timeline view can display indicators representing data selected from the group consisting of: indicators of time segments when mapping data was recorded and/or processed by system 10 (e.g. where beat grouping was performed); indicators of time segments for which cardiac activity maps have been created; indicators of when therapy was delivered to the patient; indicators of time segments when anatomy data was collected by system 10; indicators of when annotations were made to the displayed data (e.g. annotation made by a clinician via a user interface of system 10); and combinations of these.

In some embodiments, GUI 125 includes an “activation area”, as shown. The activation area can display a histogram representation of a time-referenced activation plot, such as a plot that shows the number of points and/or the area of a cardiac chamber that is active versus time. Alternatively or additionally, the activation display area can display a histogram representation of an amplitude referenced activation plot, such as a plot that shows activation amplitude range versus points and/or the area of a cardiac chamber.

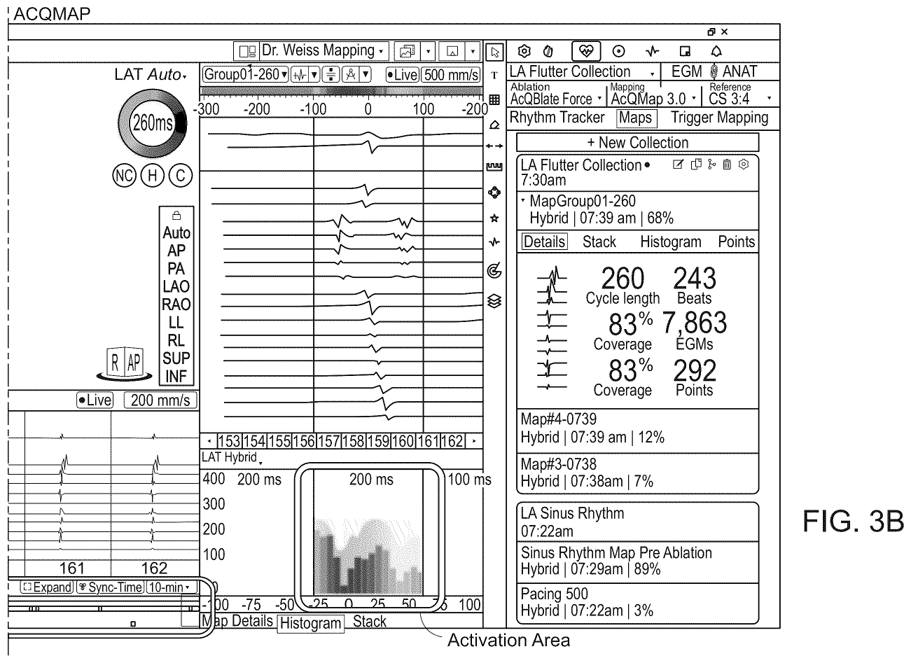

Referring now to FIGS. 4A and 4B, an example of a graphical user interface including an anatomic model, and an anatomic model including various markers, respectively, are illustrated, consistent with the present inventive concepts. System 10 can be configured to provide a graphical user interface including an anatomic model, for example GUI 125 shown. GUI 125 can include (e.g. provide) graphical data, for example cardiac activity data shown in graphical form, as shown. In FIG. 4A, a beat (e.g. a heartbeat) that has been identified by system 10 is correspondingly identified (e.g. differentiated) in the graphical data by a white box, as shown. The anatomic model can include a map of activation times (e.g. a color map of varying grayscale, as shown, and/or a color map of other varying color scheme). One or more visual markers can be displayed relative to the anatomic model, for example a visual marker indicating the point on the anatomic model where the earliest activation was recorded. In FIG. 4B, additional visual markers are displayed. In some embodiments, the size of the visual markers displayed on the anatomic model can correlate to the precision of the data represented by the marker.

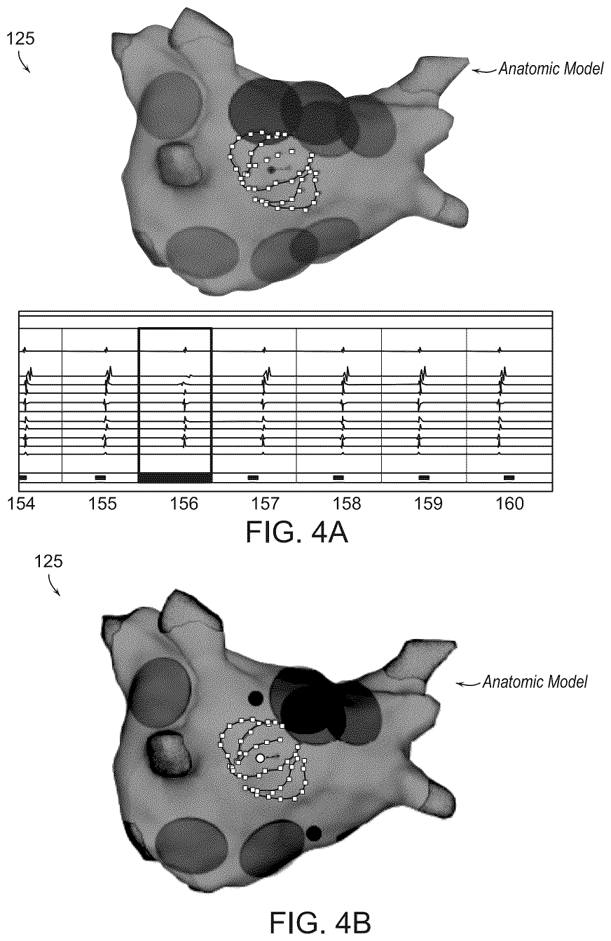

Referring now to FIGS. 5A and 5B, a flow diagram representing an embodiment of a machine learning and/or other AI-based method of rhythm classification and representations of cardiac activity data, respectively, are illustrated, consistent with the present inventive concepts. Cardiac signals (e.g. unipolar EGM signals recorded from the coronary sinus) can be recorded and categorized as representing various cardiac rhythms to be identified by a “rhythm classification algorithm” (e.g. an algorithm 115 comprising an AI algorithm or other algorithm configured to perform rhythm classification). The cardiac rhythms to be identified can be selected from the group consisting of: sinus rhythm; atrial flutter; atrial fibrillation; paced rhythm; and combinations of these. In some embodiments, the recorded signals are down sampled. The categorized groups of recorded cardiac data can be transformed by system 10 into wavelet scalograms (e.g. can be decomposed into different frequency bands while preserving temporal information). An algorithm (e.g. algorithm 115 described herein), such as an AI algorithm comprising a convolutional neural network, can be trained on the wavelet scalograms to identify cardiac rhythms. In some embodiments, the performance of the AI algorithm can be evaluated using validation data, such as to determine the classification accuracy and/or positive predictive value of the algorithm.

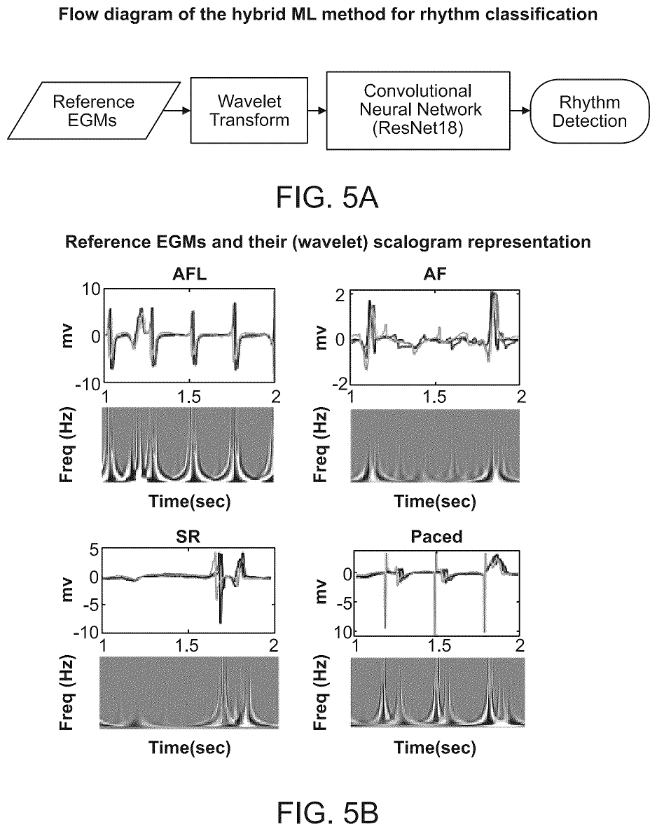

Referring now to FIG. 6, various embodiments of anatomic models upon which cardiac activity maps are displayed are illustrated, consistent with the present inventive concepts. In some embodiments, system 10 calculates conduction velocities based on cardiac activation times. A map of cardiac activation times is illustrated in the bottom left portion of FIG. 6. A map of conduction velocities is illustrated in the bottom middle portion of FIG. 6. System 10 can identify spatiotemporal patterns across cardiac tissue. These spatiotemporal patterns can be displayed relative to one or more anatomic models, as shown on the right-side portion of FIG. 6. In some embodiments, spatiotemporal patterns are displayed as colorized (e.g. using variations in greyscale as shown, or other array of color variations) histograms on the surface of the anatomic model. In the example illustrated in FIG. 6, brighter, more opaque colors represent greater prevalence of each pattern displayed. Furthermore, in the example shown, the patterns illustrated comprise focal (A), rotational (B), and complex pivoting and re-entry patterns (C).

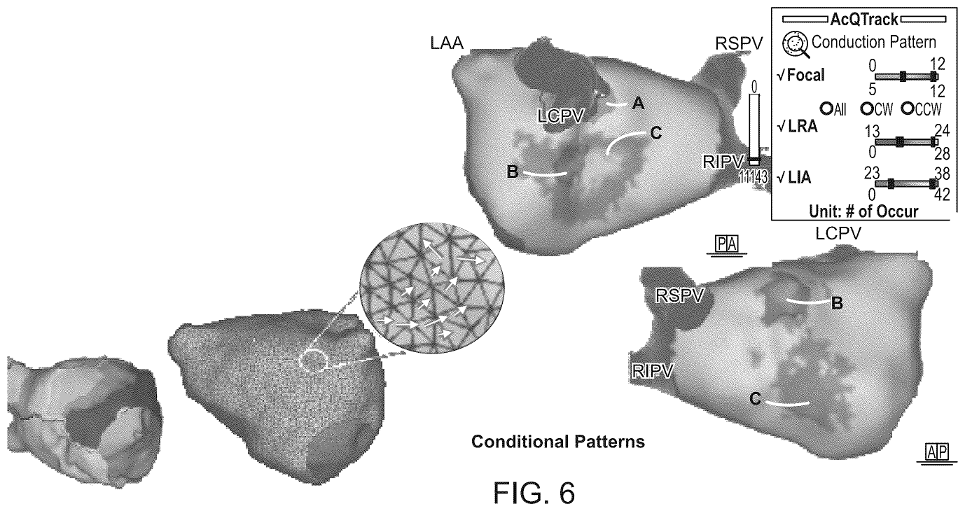

Referring now to FIG. 7, various embodiments of anatomic models upon which maps of cardiac activity are displayed are illustrated, consistent with the present inventive concepts. In some embodiments, cardiac activity maps produced by system 10 can indicate the average speed of conduction through cardiac tissue. In FIG. 7, areas of slow conduction (e.g. less than 0.3 m/s) are identified, based on data collected over 5-7 seconds, in which 50 activations occurred. In some embodiments, multiple cardiac activity maps can be aggregated by system 10 and the associated composite map can be displayed.

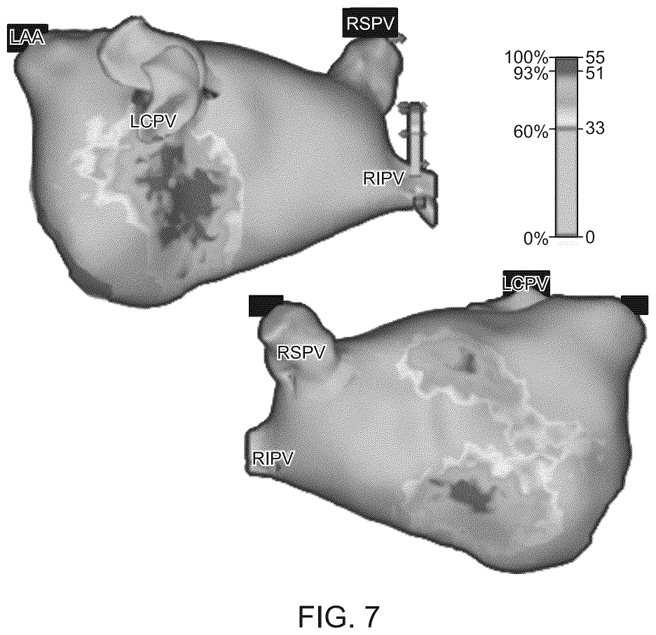

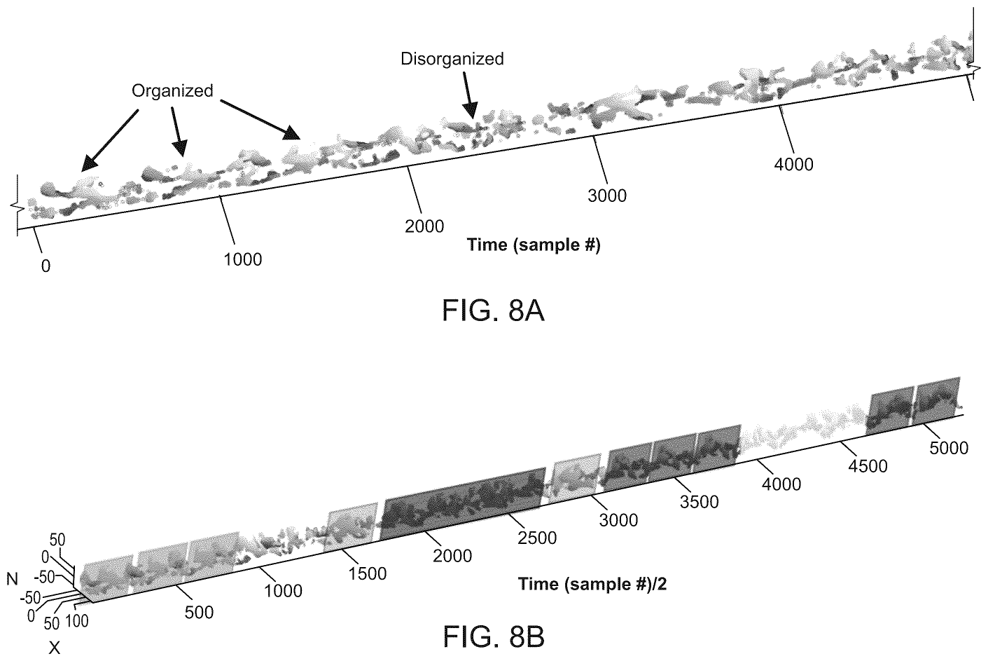

Referring now to FIG. 8A, an embodiment of a spatiotemporally-representative graph of cardiac activity is illustrated, consistent with the present inventive concepts. In FIG. 8, the long axis of the graph represents time (e.g. a time axis). In some embodiments, the graph comprises a 2D representation of a 3D map of cardiac electrical activity data (e.g. an EAM described herein). Referring additionally to FIG. 8B, an embodiment of a color-coded graph of cardiac activity is illustrated, consistent with the present inventive concepts. In some embodiments, the spatiotemporally-representative graph can be analyzed by system 10 for repetitive sequences (e.g. repetitive sequences of cardiac activity). Identified repetitive sequences can be color-coded, as shown.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A cardiac information dynamic display system, comprising:
one or more electrodes configured to record sets of electric potential data representing cardiac activity at a plurality of time intervals; and
a cardiac information console, comprising:
a signal processor configured to:
calculate sets of cardiac activity data at the plurality of time intervals using the recorded sets of electric potential data, wherein the cardiac activity data is associated with surface locations of one or more cardiac chambers; and
a user interface module configured to display information related to the cardiac activity data, the information presented relative to a graphical representation of surfaces of the one or more cardiac chambers.

2. (canceled)
